(19) United States
(12) Patent Application Publication
(10) Pub. No.: US 2022/0073942 A1
(43) Pub. Date: Mar. 10, 2022

(54) Cells with reduced inhibitor production and methods of use thereof

(71) Applicant: PFIZER INC., NEW YORK, NY (US)

(72) Inventors: Emily Ann DEUTSCHMAN, Wilmington, MA (US); Jeffrey Joseph MITCHELL, Nashua, NH (US); Bhanu Chandra MULUKUTLA, Lawrence, MA (US); John Joseph SCARCELLI, Wilmington, MA (US)

(73) Assignee: PFIZER INC., NEW YORK, NY (US)

(21) Appl. No.: 17/299,180

(22) PCT Filed: Dec. 3, 2019
(86) PCT No.: PCT/IB2019/060398

§ 371 Date: Jun. 2, 2021

Related U.S. Application Data
(60) Provisional application No. 62/776,190, filed on Dec. 6, 2018.
(60) Provisional application No. 62/932,096, filed on Nov. 7, 2019.

(51) Int. Cl. C12N 15/85; C12N 5/071

(52) U.S. Cl. CPC C12N 15/85; C12N 2510/02; C12N 5/0602

(57) ABSTRACT

The invention relates to a method of cell culture where the cells are modified to reduce the level of synthesis of growth and/or productivity inhibitors by the cell. The invention also relates to a method of cell culture for improving cell growth and productivity, in particular in culture of mammalian cells at high cell density. The invention further relates to a method of producing cells with improved cell growth and/or productivity in cell culture and to cells obtained or obtainable by such methods.

### FIELD OF THE INVENTION

The invention relates to a method of cell culture where the cells are modified to reduce the level of synthesis of growth and/or productivity inhibitors by the cell. The invention also relates to a method of cell culture for improving cell growth and productivity, in particular in culture of mammalian cells at high cell density. The invention further relates to a method of producing cells with improved cell growth and/or productivity in cell culture and to cells obtained or obtainable by such methods.

### BACKGROUND OF THE INVENTION

Proteins have become increasingly important as diagnostic and therapeutic agents. In most cases, proteins for commercial applications are produced in cell culture, from cells that have been engineered and/or selected to produce unusually high levels of a particular protein of interest. Optimization of cell culture conditions is important for successful commercial production of proteins. Mammalian cells have inefficient metabolism which causes them to consume large amounts of nutrients and convert a significant amount of them to byproducts. The byproducts are released into the culture and accumulate over the course of the culture. Lactate and ammonia, known to be the conventional inhibitors of cells in culture, are two major byproducts of cellular metabolism that accumulate to high levels in culture and beyond certain concentrations, they start inhibiting the growth and productivity of cells in culture. Cell culture methods aimed at reducing the amount of lactate and ammonia in the cell culture medium have been developed and can increase the growth and the productivity of mammalian cells. The cell growth, however, still slows down even when concentrations of lactate and ammonia are kept low, thereby limiting the maximum cell density and productivity of the cells.

Therefore, there is a need for the development of improved cell culture systems for optimum production of proteins. In particular there is a need for cell culture methods providing an increased viable cell density and/or titer and in particular for cells which are modified to reduce the level of synthesis of growth and/or productivity inhibitors.

### SUMMARY OF THE INVENTION

The invention relates to a method of cell culture comprising (i) providing cells in a cell culture medium to start a cell culture process, wherein the cells are modified to reduce the level of synthesis of growth and/or productivity inhibitors by the cell.

The invention further relates to a cell comprising one or more modified genes which reduces the level of synthesis of growth and/or productivity inhibitors by the cell, in particular wherein the inhibitor is formate or glycerol, wherein the one or more modified genes is selected from serine hydroxymethyltransferase (cytosolic) (SHMT1), serine hydroxymethyltransferase (mitochondrial) (SHMT2), C-1-tetrahydrofolate synthase (cytoplasmic) (MTHFD1), monofunctional C-1-tetrahydrofolate synthase (mitochondrial) (MTHFD1 L), bifunctional methylenetetrahydrofolate dehydrogenase/cyclohydrolase (mitochondrial) (MTHFD2), bifunctional methylenetetrahydrofolate dehydrogenase/cyclohydrolase 2-like (MTHFD2L), mitochondrial folate transporter (SLC25A32), glycerol-3-phosphate dehydrogenase (cytoplasmic) (GPD1), glycerol-3-phosphate dehydrogenase (mitochondrial) (GPD2), and phosphoglycolate phosphatase (PGP), wherein the modification increases or decreases the gene expression, and the use of the foregoing cells for the expression of a recombinant protein or polypeptide.

In some embodiments, provided herein is a method of cell culture comprising (i) providing cells in a cell culture medium to start a cell culture process, wherein the cells are modified to reduce the level of synthesis of growth or productivity inhibitors by the cell, wherein the inhibitors are formate or glycerol. Optionally, the inhibitor is formate, wherein the cells are modified to modify expression of one or more genes, and wherein the genes are selected from the group consisting of one or more of: serine hydroxymethyltransferase (cytosolic) (SHMT1), serine hydroxymethyltransferase (mitochondrial) (SHMT2), C-1-tetrahydrofolate synthase (cytoplasmic) (MTHFD1), monofunctional C-1-tetrahydrofolate synthase (mitochondrial) (MTHFD1 L), bifunctional methylenetetrahydrofolate dehydrogenase/cyclohydrolase (mitochondrial) (MTHFD2), bifunctional methylenetetrahydrofolate dehydrogenase/cyclohydrolase 2-like (MTHFD2L), and mitochondrial folate transporter (SLC25A32). Optionally, the inhibitor is formate, wherein the cells are modified to modify expression of serine hydroxymethyltransferase (mitochondrial) (SHMT2). Optionally, the inhibitor is glycerol, wherein the cells are modified to modify expression of one or more genes, and wherein the genes are selected from the group consisting of one or more of: glycerol-3-phosphate dehydrogenase (cytoplasmic) (GPD1), glycerol-3-phosphate dehydrogenase (mitochondrial) (GPD2), and phosphoglycolate phosphatase (PGP). Optionally, the inhibitor is glycerol, wherein the cells are modified to modify expression of phosphoglycolate phosphatase (PGP). Optionally, the one or more genes are modified to decrease gene expression. Optionally, the method further comprises (ii) maintaining the inhibitor formate or glycerol in the cell culture medium (a) at a concentration of no more than C2 or (b) at a concentration of at least C1 and no more than C2. Optionally, the inhibitor is formate, wherein C1 is 0.001 mM, and wherein C2 is 20 mM, 15, mM, 10 mM, 8 mM, 6 mM, 4 mM, or 2 mM. Optionally, the inhibitor is glycerol, wherein C1 is 0.001 mM, and wherein C2 is 20 mM, 15, mM, 10 mM, 8 mM 6 mM, 4 mM, or 2 mM.

In some embodiments, in a method provided herein involving step (ii) as provided above, step (ii) comprises the step of measuring the concentration of formate or glycerol, and, a) when the measured concentration is above C2, the concentration of a precursor of formate or glycerol in the cell culture medium is decreased by reducing the amount of precursor of formate or glycerol, respectively, provided to the cell culture medium, or b) when the measured concentration is below C1, the concentration of formate or glycerol in the cell culture medium is increased by adding formate or glycerol, respectively to the cell culture medium. Optionally, the concentration of formate or glycerol is measured using NMR, HPLC or UPLC, optionally online. Optionally, a pH sensor is used to monitor pH of the cell culture, and, in response to a rise above a predetermined pH value, glucose is fed to the cell culture.

In some embodiments of a method provided herein, the cell culture is a fed batch culture.

In some embodiments of a method provided herein, the cell culture method comprises a growth phase and a production phase and step (ii) is applied during the growth phase.

In some embodiments of a method provided herein, modifying the expression of the one or more genes comprises: (a) gene deletion, disruption, substitution, point mutation, multiple point mutation, insertion mutation or frameshift mutation of the gene, or (b) introduction of one or more nucleic acids comprising the one or more genes into the cell, optionally as an expressible construct or expressible vector construct.

In some embodiments, provided herein is a cell comprising one or more modified genes which reduces the level of synthesis of growth or productivity inhibitors by the cell, wherein the inhibitors are formate or glycerol. Optionally, the inhibitor is formate, wherein the cell is modified to modify expression of one or more genes, and wherein the genes are selected from the group consisting of one or more of: serine hydroxymethyltransferase (cytosolic) (SHMT1), serine hydroxymethyltransferase (mitochondrial) (SHMT2), C-1-tetrahydrofolate synthase (cytoplasmic) (MTHFD1), monofunctional C-1-tetrahydrofolate synthase (mitochondrial) (MTHFD1 L), bifunctional methylenetetrahydrofolate dehydrogenase/cyclohydrolase (mitochondrial) (MTHFD2), bifunctional methylenetetrahydrofolate dehydrogenase/cyclohydrolase 2-like (MTHFD2L), and mitochondrial folate transporter (SLC25A32). Optionally, the inhibitor is formate, wherein the cell is modified to modify expression of serine hydroxymethyltransferase (mitochondrial) (SHMT2). Optionally, the inhibitor is glycerol, wherein the cell is modified to modify expression of one or more genes, and wherein the genes are selected from the group consisting of one or more of: glycerol-3-phosphate dehydrogenase (cytoplasmic) (GPD1), glycerol-3-phosphate dehydrogenase (mitochondrial) (GPD2), and phosphoglycolate phosphatase (PGP). Optionally, the inhibitor is glycerol, wherein the cell is modified to modify expression of phosphoglycolate phosphatase (PGP). Optionally, the one or more genes are modified to decrease gene expression, and wherein gene deletion, disruption, substitution, point mutation, multiple point mutation, insertion mutation or frameshift mutation is applied to the modified gene. Optionally, the decreased gene expression results in less than or equal to any of, 90, 80, 70, 60, 50, 45, 40, 35, 30, 25, 20, 15, 10, 5, 2.5 percent level of the expression or activity of the respective modified gene as compared to the activity of the gene in a corresponding unmodified cell. Optionally, the cell is modified to modify expression of one or more genes, and wherein the genes are selected from the group consisting of one or more of: serine hydroxymethyltransferase (cytosolic) (SHMT1), serine hydroxymethyltransferase (mitochondrial) (SHMT2), C-1-tetrahydrofolate synthase (cytoplasmic) (MTHFD1), monofunctional C-1-tetrahydrofolate synthase (mitochondrial) (MTHFD1L), bifunctional methylenetetrahydrofolate dehydrogenase/cyclohydrolase (mitochondrial) (MTHFD2), bifunctional methylenetetrahydrofolate dehydrogenase/cyclohydrolase 2-like (MTHFD2L), and mitochondrial folate transporter (SLC25A32), and wherein the concentration of formate in the media of a culture of the cell is less than is 20 mM, 15, mM, 10 mM, 8 mM 6 mM, 4 mM, or 2 mM at Day 14, 13, 12, 11, 10, 9, 8, 7, or 6 of the culture, wherein inoculation of the cell culture is on Day 0. Optionally, the cell is modified to modify expression of the gene serine hydroxymethyltransferase (cytosolic) (SHMT2), and wherein the concentration of formate in the media of a culture of the cell is less than is 20 mM, 15, mM, 10 mM, 8 mM 6 mM, 4 mM, or 2 mM at Day 14, 13, 12, 11, 10, 9, 8, 7, or 6 of the culture, wherein inoculation of the cell culture is on Day 0. Optionally, the cell is modified to modify expression of one or more genes, and wherein the genes are selected from the group consisting of one or more of: glycerol-3-phosphate dehydrogenase (cytoplasmic) (GPD1), glycerol-3-phosphate dehydrogenase (mitochondrial) (GPD2), and phosphoglycolate phosphatase (PGP), and wherein the concentration of glycerol in the media of a culture of the cell is less than is 20 mM, 15, mM, 10 mM, 8 mM 6 mM, 4 mM, or 2 mM at Day 14, 13, 12, 11, 10, 9, 8, 7, or 6 of the culture, wherein inoculation of the cell culture is on Day 0. Optionally, the cell is modified to modify expression of the gene phosphoglycolate phosphatase (PGP), and wherein the concentration of glycerol in the media of a culture of the cell is less than is 20 mM, 15, mM, 10 mM, 8 mM 6 mM, 4 mM, or 2 mM at Day 14, 13, 12, 11, 10, 9, 8, 7, or 6 of the culture, wherein inoculation of the cell culture is on Day 0. Optionally, the cell is modified to reduce expression of the gene phosphoglycolate phosphatase (PGP), wherein the cell has a reduced level of synthesis of lactate as compared to a corresponding unmodified cell. Optionally, the cell is modified to modify expression of the gene phosphoglycolate phosphatase (PGP), and wherein the concentration of lactate in the media of a culture of the cell is less than is 20 mM, 15, mM, 10 mM, 8 mM 6 mM, 4 mM, or 2 mM at Day 14, 13, 12, 11, 10, 9, 8, 7, or 6 of the culture, wherein inoculation of the cell culture is on Day 0. Optionally, the cell is modified to reduce expression of the gene phosphoglycolate phosphatase (PGP), wherein the cell has a reduced level of synthesis of both lactate and glycerol as compared to a corresponding unmodified cell.

In some embodiments of a method or cell provided herein, the cells are CHO cells.

In some embodiments of a method or cell provided herein, the cells express a heterologous recombinant protein. Optionally, a method provided herein further comprises obtaining and purifying the recombinant protein produced by the cells.

In some embodiments of a method or cell provided herein, cell growth or productivity are increased as compared to a control culture, said control culture being identical except comprising unmodified cells. Optionally, cell growth is determined by maximum viable cell density and is increased by at least 5% as compared to the control culture. Optionally, productivity is determined by titer of expressed recombinant protein and is increased by at least 5% as compared to the control culture. Optionally, the maximum viable cell density of the cell culture is greater than 1×10⁶cells/mL, 5×10⁶cells/mL, 1×10⁷cells/mL, 5×10⁷ cells/mL, 1×10⁸ cells/mL or 5×10⁸ cells/mL.

In some embodiments provided herein involving a cell comprising one or more modified genes which reduces the level of synthesis of formate or glycerol, the cell further contains a modification in one or more genes selected from Bcat1, Bcat2, Bckdha/b, Dbt/Dld, lvd, Acadm, Mccc1, Mccc2, Auh, Hmgcl, Fasn, Pah, PCBD1, QDPR, Mif, Got1, Got2, Nup62-il4i1, Hpd, Hgd, Gstz1, and Fah. Optionally, the gene is Bcat1. Optionally the modification is to decrease gene expression. Optionally the modification is to increase gene expression. Optionally the gene is Bcat1, and the modification is to decrease gene expression. In some embodiments provided herein involving a cell comprising one or more modified genes which reduces the level of synthesis of formate or glycerol, the cell further contains a modification in one or more genes as described in WO2017/051347, which is hereby incorporated by reference for all purposes.

### BRIEF DESCRIPTION OF DRAWINGS

FIG. 1A shows the levels of cell density, format, and glycerol in a conventional fed-batch mammalian cell culture over time. The X-axis is time (day) of the cell culture, and the Y-axis is viable cell density (in 10⁶ cells/ml) or metabolite concentration. The graphed values are ammonia (mM) (diamonds), lactate (g/L)(triangles), and cell density (squares).

FIG. 1B shows the levels of cell density, format, and glycerol in a glucose-restricted fed-batch (HIPDOG) mammalian cell culture over time. The X-axis is time (day) of the cell culture, and the Y-axis is viable cell density (in 10⁶ cells/ml) or metabolite concentration. The graphed values are ammonia (mM) (diamonds), lactate (g/L)(triangles), and cell density (squares).

FIG. 2A shows the level of formate in either a conventional fed-batch mammalian cell culture (squares) or a glucose-restricted fed-batch (HIPDOG) mammalian cell culture (diamonds) over time. The X-axis is time (day) of the cell culture, and the Y-axis is formate concentration (mM).

FIG. 2B shows the level of glycerol in either a conventional fed-batch mammalian cell culture (squares) or a glucose-restricted fed-batch (HIPDOG) mammalian cell culture (diamonds) over time. The X-axis is time (day) of the cell culture, and the Y-axis is formate concentration (mM).

FIG. 3A shows the effect of various concentrations of glycerol on viable cell density (VCD) of CHO cells. Specifically, the viable cell density of CHO cultures supplemented with either 0, 2, or 4 mM glycerol are shown (from left to right; each glycerol concentration has VCD values shown at days 0, 3, and 6 of culture). The viable cell density is shown on the Y-axis (in 10⁶ cells/ml).

FIG. 3B shows the effect of various concentrations of formate on viable cell density (VCD) of CHO cells. Specifically, the viable cell density of CHO cultures supplemented with either 0, 2, 4, or 6 mM formate are shown (from left to right; each glycerol concentration has VCD values shown at days 0, 4, and 5 of culture). The viable cell density is shown on the Y-axis (in 10⁶ cells/ml).

FIG. 4A shows the level of serine in a standard fed-batch culture process and in a low amino acid-fed batch culture process. The X-axis is time (day) of the cell culture, and the Y-axis is serine concentration (mM). The graphed values are the serine concentration in the standard fed-batch culture (diamonds) or the low amino acid culture (squares).

FIG. 4B shows the level of formate in the standard fed-batch culture process and in a low amino acid-fed batch culture process shown in FIG. 4A. The X-axis is time (day) of the cell culture, and the Y-axis is formate concentration (mM). The graphed values are the formate concentration in the standard fed-batch culture (diamonds) or the low amino acid culture (squares).

FIG. 5 shows aspects of the formate metabolism pathway.

FIG. 6 shows aspects of the glycerol metabolism pathway.

FIG. 7 shows immunoblots of lysates of i) various clonal cell lines subject to CRISPR gene editing of the PGP gene (clones 377, 406, 343, 609, 878) ii) a control clone (clone 217) and iii) the untransfected (host) cell line. The lysates were probed with an anti-PGP antibody (top panel) and an anti-B-actin antibody (bottom panel; serves as a loading control).

FIG. 8A shows the concentration of glycerol over time in spent media from the cultures of the various clones as indicated (solid circle: full KO PGP clones 343, 609, 878; solid square: partial KO PGP clones 377 and 406; open square, dashed line: control clone 217; open square, solid line: host cell). (While separate lines are provided for the different KO clones, in this and other figures herein, there is significant overlap between each of the full KO clones so they often appear as one line, and there is significant overlap between of the partial KO clones so they often appear as one line). 8B shows specific glycerol production over time (picograms (pcg) per cell per day) of the various clones as indicated (solid circle: full KO PGP clones 343, 609, 878; solid square: partial KO PGP clones 377 and 406; open square, dashed line: control clone 217; open square, solid line: host cell).

FIG. 9 shows the concentration of lactate over time in spent media from the cultures of the various clones as indicated (solid circle: full KO PGP clones 343, 609, 878; solid square: partial KO PGP clones 377 and 406; open square, dashed line: control clone 217; open square, solid line: host cell).

FIG. 10 shows immunoblots of lysates of i) various clonal cell lines subject to CRISPR gene editing of the SHMT2 gene (clones 277, 482, 746) ii) a control clone (clone 425) and iii) the untransfected (host) cell line. The lysates were probed with an anti-SHMT2 antibody (top panel) and an anti-B-actin antibody (bottom panel; serves as a loading control).

FIG. 11 shows the concentration of formate over time in spent media from the cultures of the various clones as indicated (solid square: SHMT2 KO clones 746 and 277; solid circle, dashed line: control clone 425; solid circle, solid line: host cell).

### DETAILED DESCRIPTION

In some embodiments, provided herein are cells, methods and media for cell culture.

In some embodiments, the present invention provides cell culture methods where the concentration of at least one metabolite selected from formate and glycerol is maintained at low levels in the cell culture medium.

The inventors have unexpectedly discovered that, in cell culture, and in particular in high density cell culture, such as for example fed-batch cell culture aiming at producing high amount of a recombinant protein of interest, the growth of cells was inhibited by the accumulation of metabolites such as formate and glycerol in the cell culture medium. The inhibitory effect of these metabolites can be limited by maintaining their concentration in the cell culture medium below levels where they inhibit cell growth. These inhibitory metabolites (formate and glycerol) may also be considered (and referred to herein) as growth and/or productivity inhibitors. The inhibitory effects formate and glycerol can also be can be limited by modifying one or more genes in the cell to reduce the level of synthesis of formate or glycerol by the cell, in particular where the one or more modified genes is selected from serine hydroxymethyltransferase (cytosolic) (SHMT1), serine hydroxymethyltransferase (mitochondrial) (SHMT2), C-1-tetrahydrofolate synthase (cytoplasmic) (MTHFD1), monofunctional C-1-tetrahydrofolate synthase (mitochondrial) (MTHFD1L), bifunctional methylenetetrahydrofolate dehydrogenase/cyclohydrolase (mitochondrial) (MTHFD2), bifunctional methylenetetrahydrofolate dehydrogenase/cyclohydrolase 2-like (MTHFD2L), mitochondrial folate transporter (SLC25A32), glycerol-3-phosphate dehydrogenase (cytoplasmic) (GPD1), glycerol-3-phosphate dehydrogenase (mitochondrial) (GPD2), and phosphoglycolate phosphatase (PGP). Optionally, one or more of the genes SHMT1, SHMT2, MTHFD1, MTHFD1 L, MTHFD2, MTHFD2L, and SLC25A32 is modified to reduce the level of formate produced by the cell. Optionally, one or more of the genes GPD1, GPD2, and PGP is modified to reduce the level of glycerol produced by the cell.

Serine Hydroxymethyltransferase, Cytosolic (SHMT1)

Serine hydroxymethyltransferase (SHMT1) is a cytosolic enzyme that catalyzes the reversible conversion of serine and tetrahydrofolate to glycine and 5,10-methylene tetrahydrofolate. Exemplary SHMT1 amino acid sequences are provided under UniProt ID numbers: P50431 (mouse), Q6TXG7 (rat), and P34896 (human).

Serine hydroxymethyltransferase, mitochondrial (SHMT2)

Serine hydroxymethyltransferase (SHMT2) is a mitochondrial enzyme that catalyzes the reversible conversion of serine and tetrahydrofolate to glycine and 5,10-methylene tetrahydrofolate. Exemplary SHMT2 amino acid sequences are provided under UniProt ID numbers: Q9CZN7 (mouse), Q5U3Z7 (rat), and P34897 (human).

C-1-tetrahydrofolate synthase (cytoplasmic) (MTHFD1)

C-1-tetrahydrofolate synthase (cytoplasmic)/MTHFD1 (also known as methylenetetrahydrofolate dehydrogenase (NADP+dependent)) is an enzyme involved in tetrahydrofolate interconversion (e.g. 5,10-methylenetetrahydrofolate+NADP⁺ to 5,10-methenyltetrahydrofolate+NADPH). Exemplary MTHFD1 amino acid sequences are provided under UniProt ID numbers: Q922D8 (mouse), P27653 (rat), and P11586 (human).

Monofunctional C-1-tetrahydrofolate synthase (mitochondrial) (MTHFD1L) Monofunctional C-1-tetrahydrofolate synthase (mitochondrial)/MTHFD1L (also known as methylenetetrahydrofolate dehydrogenase (NADP+ dependent) 1-like) is an enzyme that has formyltetrahydrofolate synthetase activity. Exemplary MTHFD1 L amino acid sequences are provided under UniProt ID numbers: Q3V3R1 (mouse), B2GUZ3 (rat), and Q6UB35 (human).

Bifunctional methylenetetrahydrofolate dehydrogenase/cyclohydrolase (mitochondrial) (MTHFD2)

Bifunctional methylenetetrahydrofolate dehydrogenase/cyclohydrolase (mitochondrial)/MTHFD2 (also known as methylenetetrahydrofolate dehydrogenase (NAD+ dependent)) is an enzyme involved in tetrahydrofolate interconversion (e.g. 5,10-methylenetetrahydrofolate+NAD⁺ to 5,10-methenyltetrahydrofolate+ NADH). Exemplary MTHFD2 amino acid sequences are provided under UniProt ID numbers: P18155 (mouse), D4A1Y5 (rat), and P13995 (human).

Bifunctional methylenetetrahydrofolate dehydrogenase/cyclohydrolase 2-Like (MTHFD2L)

Bifunctional methylenetetrahydrofolate dehydrogenase/cyclohydrolase 2-like/MTHFD2L (also known as methylenetetrahydrofolate dehydrogenase (NADP+dependent) 2-like) is an enzyme involved in tetrahydrofolate interconversion (e.g. 5,10-methylenetetrahydrofolate+NAD⁺ to 5,10-methenyltetrahydrofolate+ NADH). Exemplary MTHFD2L amino acid sequences are provided under UniProt ID numbers: D3YZG8 (mouse), D3ZUAO (rat), and Q9H903 (human).

Mitochondrial folate transporter (SLC25A32)

Mitochondrial folate transporter/SLC25A32 (also known as solute carrier family 25, member 32) is an enzyme involved in the transport of folate across the inner membranes of mitochondria. Exemplary SLC25A32 amino acid sequences are provided under UniProt ID numbers: Q8BMG8 (mouse), B2GV53 (rat), and Q9H2D1 (human).

Glycerol-3-phosphate fehydrogenase (cytoplasmic) (GPD1)

Glycerol-3-phosphate dehydrogenase/GPD1 (also known as glycerol-3-phosphate dehydrogenase 1 (soluble)) is a cytoplasmic enzyme that has NAD⁺ dependent glycerol 3-phosphate dehydrogenase activity. Exemplary GPD1 amino acids sequences are provided under UniProt ID numbers: P13707 (mouse), O35077 (rat), and P21695 (human).

Glycerol-3-phosphate dehydrogenase (mitochondrial) (GPD2)

Glycerol-3-phosphate dehydrogenase/GPD2 (also known as glycerol phosphate dehydrogenase 2, mitochondrial) is a mitochondrial enzyme that has FAD⁺ dependent glycerol 3-phosphate dehydrogenase activity. Exemplary GPD2 amino acids sequences are provided under UniProt ID numbers: Q64521 (mouse), P35571 (rat), and P43304 (human).

Phosphoglycolate phosphatase (PGP)

Phosphoglycolate phosphatase/PGP is also known as “glycerol-3-phosphate phosphatase” (G3PP) and “aspartate-based ubiquitous Mg(2+)-dependent phosphatase” (AUM). It has phosphatase activity and hydrolyzes glycerol-3-phosphate into glycerol. Exemplary PGP amino acids sequences are provided under UniProt ID numbers: Q8CHP8 (mouse), D3ZDK7 (rat), and A6NDG6 (human).

For each of the genes listed above, the corresponding gene in other organisms (e.g. Chinese hamster/Cricetulus griseus) can be readily identified by a person of skill in the art based on, for example, sequence homology of a gene in the organism of interest with a sequence provided above. For example, Chinese hamster genes are available from CHOGeneome.org or KEGG Genome, or may be identified via a BLAST search via the US National Center for Biotechnology Information (NCBI) BLAST searching tool.

Methods Comprising Controlling the Metabolite Concentration in the Cell Culture Medium at Low Levels

In one aspect the invention provides a method of cell culture comprising (i) providing cells in a cell culture medium to start a cell culture process, wherein the cells are modified to reduce the level of synthesis of growth and/or productivity inhibitors by the cell. In some embodiments, the growth and/or productivity inhibitors are formate and/or glycerol.

In some embodiments, the cells are modified to modulate expression of one or more genes in a cell metabolic pathway or pathways which are involved in formate metabolism (e.g. formate synthesis or formate catabolism). In some embodiments, the cells are modified to modulate expression of one or more genes involved in formate metabolism, wherein the gene is selected from SHMT1, SHMT2, MTHFD1, MTHFD1 L, MTHFD2, MTHFD2L, and SLC25A32. In some embodiments, the cells are modified to decrease the generation of formate by the cell, wherein the expression of one or more of the genes SHMT1, SHMT2, MTHFD1, MTHFD1L, MTHFD2, MTHFD2L, and SLC25A32 is reduced. In some embodiments, the cells are modified to decrease the generation of formate by the cell, wherein the expression of one or more of the genes SHMT1, SHMT2, MTHFD1, MTHFD1L, MTHFD2, MTHFD2L, and SLC25A32 is increased.

In some embodiments, the cells are modified to modulate expression of one or more genes in a cell metabolic pathway or pathways which are a branching pathway relating to serine metabolism, and as a result to reduce the generation of formate production by the cell. Optionally, the genes may be selected from one or more of MTHFR, TYMS, MTFMT, and GART/ATIC. In some embodiments, the cells are modified to decrease the generation of formate by the cell, wherein the expression of one or more of the genes MTHFR, TYMS, MTFMT, and GART/ATIC is reduced. In some embodiments, the cells are modified to decrease the generation of formate by the cell, wherein the expression of one or more of the genes MTHFR, TYMS, MTFMT, and GART/ATIC is increased.

In some embodiments, the cells are modified to modulate expression of one or more genes in a cell metabolic pathway or pathways which are involved in glycerol metabolism (e.g. glycerol synthesis or glycerol catabolism). In some embodiments, the cells are modified to modulate expression of one or more genes involved in glycerol metabolism, wherein the gene is selected from GPD1, GPD2, and PGP. In some embodiments, the cells are modified to decrease the generation of formate by the cell, wherein the expression of one or more of the genes GPD1, GPD2, and PGP is reduced. In some embodiments, the cells are modified to decrease the generation of formate by the cell, wherein the expression of one or more of the genes GPD1, GPD2, and PGP is increased.

In some embodiments, a modified cell provided herein comprises an expressible nucleic acid or vector construct comprising a SHMT1, SHMT2, MTHFD1, MTHFD1L, MTHFD2, MTHFD2L, SLC25A32, GPD1, GPD2, or PGP gene.

In some embodiments a method provided herein comprises maintaining glycerol or formate below a concentration C2 in the cell culture medium, wherein C2 is 2 mM. In some embodiments, C2 is 50 mM, 20 mM 30 mM, 20 mM, 15, mM, 10 mM, 8 mM 6 mM, 4 mM, 2.5 mM, 2 mM, 1.5 mM, 1 mM, 0.9 mM, 0.8 mM, 0.7 mM, 0.6 mM, 0.5 mM, 0.4 mM, 0.3 mM, 0.2 mM, 0.1 mM, 0.05 mM, 0.04 mM, 0.03 mM, 0.02 mM, 0.01 mM, 0.005 mM, 0.004 mM, 0.003 mM, 0.002 mM or 0.001 mM. In some embodiments, C2 is 6 mM. In some embodiments, C2 is 0.4 mM. In some embodiments, C2 is 1 mM. In some embodiments, C2 is 0.5 mM.

In some embodiments, a method provided herein comprises the step of measuring the concentration of at least one of formate or glycerol

In some embodiments, when the measured concentration is above a predefined value, the concentration of precursor of at least one of formate or glycerol in the cell culture medium is decreased by reducing the amount of precursor provided to the cells. Said predefined value is selected so that the decrease of concentration of said precursor prevents the concentration of formate or glycerol from rising above C2. The predefined value can be equal to C2 or can be a percentage of C2. In some embodiments the percentage is 50, 55, 60, 65, 70, 75, 80, 85, 90 or 95% of C2. In some embodiments the percentage is 80% of C2.

Concentration of lactate and ammonia

In some embodiments of, other metabolites inhibiting growth of cells, such as lactate and ammonia are also maintained at low levels in the cell culture medium. Methods to keep lactate and ammonia at low levels are known to the skilled person.

For example, lactate can be kept at low levels in cell culture by using methods disclosed in WO2004104186, Gagnon et al, Biotechnology and Bioengineering, Vol. 108, No. 6, June, 2011 (Gagnon et A1) or WO2004048556.

Cell Culture Methods

The terms “culture” and “cell culture” as used herein refer to a cell population that is suspended in a medium under conditions suitable to survival and/or growth of the cell population. As will be clear to those of ordinary skill in the art, in some embodiments, these terms as used herein refer to the combination comprising the cell population and the medium in which the population is suspended. In some embodiments, the cells of the cell culture comprise mammalian cells.

The present invention may be used with any cell culture method that is amenable to the desired process (e.g., production of a recombinant protein (e.g., antibody)). As a non-limiting example, cells may be grown in batch or fed-batch cultures, where the culture is terminated after sufficient expression of the recombinant protein (e.g., antibody), after which the expressed protein (e.g., antibody) is harvested. Alternatively, as another non-limiting example, cells may be grown in batch-refeed, where the culture is not terminated and new nutrients and other components are periodically or continuously added to the culture, during which the expressed recombinant protein (e.g., antibody) is harvested periodically or continuously. Other suitable methods (e.g., spin-tube cultures) are known in the art and can be used to practice the present invention.

In some embodiments, a cell culture suitable for the present invention is a fed-batch culture. The term “fed-batch culture” as used herein refers to a method of culturing cells in which additional components are provided to the culture at a time or times subsequent to the beginning of the culture process. Such provided components typically comprise nutritional components for the cells which have been depleted during the culturing process. A fed-batch culture is typically stopped at some point and the cells and/or components in the medium are harvested and optionally purified. In some embodiments, the fed-batch culture comprises a base medium supplemented with feed media.

Cells may be grown in any convenient volume chosen by the practitioner. For example, cells may be grown in small scale reaction vessels ranging in volume from a few milliliters to several liters. Alternatively, cells may be grown in large scale commercial Bioreactors ranging in volume from approximately at least 1 liter to 10, 50, 100, 250, 500, 1000, 2500, 5000, 8000, 10,000, 12,000, 15000, 20000 or 25000 liters or more, or any volume in between.

The temperature of a cell culture will be selected based primarily on the range of temperatures at which the cell culture remains viable and the range in which a high level of desired product (e.g., a recombinant protein) is produced. In general, most mammalian cells grow well and can produce desired products (e.g., recombinant proteins) within a range of about 25° C. to 42° C., although methods taught by the present disclosure are not limited to these temperatures. Certain mammalian cells grow well and can produce desired products (e.g., recombinant proteins or antibodies) within the range of about 35° C. to 40° C. In certain embodiments, a cell culture is grown at a temperature of 20, 21, 22, 23, 24, 25, 26, 27, 28, 29, 30, 31, 32, 33, 34, 35, 36, 37, 38, 39, 40, 41, 42, 43, 44 or 45° C. at one or more times during the cell culture process. Those of ordinary skill in the art will be able to select appropriate temperature or temperatures in which to grow cells, depending on the particular needs of the cells and the particular production requirements of the practitioner. The cells may be grown for any amount of time, depending on the needs of the practitioner and the requirement of the cells themselves. In some embodiment, the cells are grown at 37° C. In some embodiments, the cells are grown at 36.5° C.

In some embodiments, the cells may be grown during the initial growth phase (or growth phase) for a greater or lesser amount of time, depending on the needs of the practitioner and the requirement of the cells themselves. In some embodiments, the cells are grown for a period of time sufficient to achieve a predefined cell density. In some embodiments, the cells are grown for a period of time sufficient to achieve a cell density that is a given percentage of the maximal cell density that the cells would eventually reach if allowed to grow undisturbed. For example, the cells may be grown for a period of time sufficient to achieve a desired viable cell density of 1, 5, 10, 15, 20, 25, 30, 35, 40, 45, 50, 55, 60, 65, 70, 75, 80, 85, 90, 95 or 99 percent of maximal cell density. In some embodiments, the cells are grown until the cell density does not increase by more than 15%, 14%, 13%, 12%, 11%, 10%, 9%, 8%, 7%, 6%, 5%, 4%, 3%, 2% or 1% per day of culture. In some embodiments, the cells are grown until the cell density does not increase by more than 5% per day of culture.

In some embodiment the cells are allowed to grow for a defined period of time. For example, depending on the starting concentration of the cell culture, the temperature at which the cells are grown, and the intrinsic growth rate of the cells, the cells may be grown for 0, 1, 2, 3, 4, 5, 6, 7, 8, 9, 10, 11, 12, 13, 14, 15, 16, 17, 18, 19, 20 or more days, preferably for 4 to 10 days. In some cases, the cells may be allowed to grow for a month or more. The practitioner of the present invention will be able to choose the duration of the initial growth phase depending on protein production requirements and the needs of the cells themselves.

The cell culture may be agitated or shaken during the initial culture phase in order to increase oxygenation and dispersion of nutrients to the cells. In accordance with the present invention, one of ordinary skill in the art will understand that it can be beneficial to control or regulate certain internal conditions of the bioreactor during the initial growth phase, including but not limited to pH, temperature, oxygenation, etc.

At the end of the initial growth phase, at least one of the culture conditions may be shifted so that a second set of culture conditions is applied and a metabolic shift occurs in the culture. A metabolic shift can be accomplished by, e.g., a change in the temperature, pH, osmolality or chemical inductant level of the cell culture. In one non-limiting embodiment, the culture conditions are shifted by shifting the temperature of the culture. However, as is known in the art, shifting temperature is not the only mechanism through which an appropriate metabolic shift can be achieved. For example, such a metabolic shift can also be achieved by shifting other culture conditions including, but not limited to, pH, osmolality, and sodium butyrate levels. The timing of the culture shift will be determined by the practitioner of the present invention, based on protein production requirements or the needs of the cells themselves.

When shifting the temperature of the culture, the temperature shift may be gradual. For example, it may take several hours or days to complete the temperature change. Alternatively, the temperature shift may be abrupt. For example, the temperature change may be complete in less than several hours. Given the appropriate production and control equipment, such as is standard in the commercial large-scale production of polypeptides or proteins, the temperature change may even be complete within less than an hour.

In some embodiments, once the conditions of the cell culture have been shifted as discussed above, the cell culture is maintained for a subsequent production phase under a second set of culture conditions conducive to the survival and viability of the cell culture and appropriate for expression of the desired polypeptide or protein at commercially adequate levels.

As discussed above, the culture may be shifted by shifting one or more of a number of culture conditions including, but not limited to, temperature, pH, osmolality, and sodium butyrate levels. In some embodiments, the temperature of the culture is shifted. According to this embodiment, during the subsequent production phase, the culture is maintained at a temperature or temperature range that is lower than the temperature or temperature range of the initial growth phase. As discussed above, multiple discrete temperature shifts may be employed to increase cell density or viability or to increase expression of the recombinant protein.

In some embodiments, the cells may be maintained in the subsequent production phase until a desired cell density or production titer is reached. In another embodiment of the present invention, the cells are allowed to grow for a defined period of time during the subsequent production phase. For example, depending on the concentration of the cell culture at the start of the subsequent growth phase, the temperature at which the cells are grown, and the intrinsic growth rate of the cells, the cells may be grown for 1, 2, 3, 4, 5, 6, 7, 8, 9, 10, 11, 12, 13, 14, 15, 16, 17, 18, 19, 20 or more days. In some cases, the cells may be allowed to grow for a month or more. The practitioner of the present invention will be able to choose the duration of the subsequent production phase depending on polypeptide or protein production requirements and the needs of the cells themselves.

The cell culture may be agitated or shaken during the subsequent production phase in order to increase oxygenation and dispersion of nutrients to the cells. In accordance with the present invention, one of ordinary skill in the art will understand that it can be beneficial to control or regulate certain internal conditions of the bioreactor during the subsequent growth phase, including but not limited to pH, temperature, oxygenation, etc.

In some embodiments, the cells express a recombinant protein and the cell culture method of the invention comprises a growth phase and a production phase.

In some embodiments of a method of cell culture provided herein (e.g. which includes a first step of (i) providing cells in a cell culture medium to start a cell culture process, wherein the cells are modified to reduce the level of synthesis of growth and/or productivity inhibitors by the cell), the method further comprises (ii) maintaining the inhibitor formate or glycerol in the cell culture medium (a) at a concentration of no more than C2 or (b) at a concentration of at least C1 and no more than C2. In some embodiments, C2 is 0.005, 0.01, 0.05, 0.1, 0.2, 0.5, 1, 2, 5, or 10 mM. In some embodiments, Cl is 0.001, 0.005, 0.01, 0.05, 0.1, 0.2, 0.5, 1, 2, or 5 mM, and C2 is 0.005, 0.01, 0.05, 0.1, 0.2, 0.5, 1, 2, 5, or 10 mM, wherein C2 is a larger value than C1. In some embodiments of the method of cell culture, step (ii) is applied during the totality of the cell culture method. In some embodiments step (ii) is applied during a part of the cell culture method. In some embodiments, step (ii) is applied until a predetermined viable cell density is obtained. In some embodiments, the cell culture method of the invention comprises a growth phase and a production phase and step (ii) is applied during the growth phase. In some embodiments, the cell culture method of the invention comprises a growth phase and a production phase and step (ii) is applied during a part of the growth phase. In some embodiments, the cell culture method of the invention comprises a growth phase and a production phase and step (ii) is applied during the growth phase and the production phase. In step (ii), the term “maintaining” can refer to maintaining the concentration of amino acid or metabolite above or below a certain level for the entire culture process (until harvesting) or for a part of the culture process such as for example the growth phase, a part of the growth phase or until a predetermined cell density is obtained.

In one embodiment gene knockdown reduces gene expression or activity or activity of the encoded molecule (e.g. protein) to less than or equal to any of, 90, 80, 70, 60, 50, 45, 40, 35, 30, 25, 20, 15, 10, 5, 2.5 percent level of the expression or activity of the encoded molecule as compared to in unmodified cells.

In some embodiments, SHMT1, SHMT2, MTHFD1, MTHFD1L, MTHFD2, MTHFD2L, SLC25A32, GPD1, GPD2, or PGP gene knockdown reduces gene expression or activity or activity of the encoded molecule (e.g. protein) to less than or equal to any of, 90, 80, 70, 60, 50, 45, 40, 35, 30, 25, 20, 15, 10, 5, 2.5 percent level of SHMT1, SHMT2, MTHFD1, MTHFD1L, MTHFD2, MTHFD2L, SLC25A32, GPD1, GPD2, or PGP expression or activity of the encoded molecule as compared to in unmodified cells.

Knockdown can be achieved by, for example, any one or more of gene deletion, disruption, substitution, point mutation, multiple point mutation, insertion mutation or frameshift mutation applied to the identified gene to be expressed at a decreased level or by repression of gene expression by use of CRISPR/CAS9 or CRISPR interference or interfering RNA, interfering mRNA or interfering aptamer, or siRNA or siRNA interference or a zinc finger transcription factor or a zinc finger nuclease or a transcription activator-like effector nucleases (TALEN) or by use of an inhibitor such as a an inhibitor molecule or small molecule inhibitor, for example an activity inhibitor of protein or enzyme activity.

In some embodiments, reducing the activity of a gene may involve knockdown of a gene in a host cell (e.g. a CHO cell) by any of the methods described above, and then by further introducing an orthologous corresponding gene from a different species into the host cell. For example, after knockdown of SHMT2 gene in a CHO cell, an orthologous SHMT2 gene from a fish may be introduced into the CHO cell (e.g. UniProt Reference No. A9LDD9; zebrafish SHMT2), wherein the zebrafish SHMT2 protein is active in the CHO cell, but has reduced SHMT2 activity as compared to the endogenous SHMT2 protein.

According to some embodiments the modification suppresses, reduces, or prevents the biosynthesis of the growth and/or productivity inhibitor and/or an intermediate thereof, in some embodiments the modification suppresses, reduces, and prevents the biosynthesis of the growth and/or productivity inhibitor. According to some embodiments the modification produces cells with improved cell growth and/or productivity in cell culture.

In some embodiments modifying the expression of the one or more genes comprises;

(a) any one or more of gene deletion, disruption, substitution, point mutation, multiple point mutation, insertion mutation or frameshift mutation applied to the identified gene which is expressed at an increased or decreased level or identified gene which comprises a mutation, or

(b) introduction of one or more nucleic acids comprising the gene into the cell, optionally as an expressible nucleic acid or vector construct,

(c) repression or activation of gene expression by use of CRISPR/CAS9 or CRISPR interference or interfering RNA, interfering mRNA or interfering aptamer, or siRNA or siRNA interference or a zinc finger transcription factor or a zinc finger nuclease or a transcription activator-like effector nucleases (TALEN).

Where modifying the expression of the one or more genes comprises use of an interfering RNA (RNAi), suitable RNAi include RNAi that decreases or increases the level of a gene product, i.e. targets the one or more genes. For example, an RNAi can be an shRNA or siRNA. A “small interfering” or “short interfering RNA” or siRNA is a RNA duplex of nucleotides that is targeted to a gene interest or the one or more genes. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. A “short hairpin RNA,” or shRNA, is a polynucleotide construct that can be made to express an interfering RNA such as siRNA.

In some embodiments the vector contains one or more of a promoter sequence, a directional cloning site, an epitope tag, a polyadenylation sequence, and antibiotic resistance gene. In some embodiments the promoter sequence is Human cytomegalovirus immediate early promoter, the directional cloning site is TOPO, the epitope tag is V5 for detection using anti-V5 antibodies, the polyadenylation sequence is from Herpes Simplex Virus thymidine kinase, and antibiotic resistance gene is Blasticidin.

In some embodiments of the preceding embodiments the one or more genes is modified to increase gene expression, in some embodiments by mutation of the gene, in some embodiments by introduction of a copy of the wild type gene into the cell optionally as an expressible vector.

In some embodiments the gene expression of SHMT1, SHMT2, MTHFD1, MTHFD1 L, MTHFD2, MTHFD2L, SLC25A32, GPD1, GPD2, or PGP is reduced, either by gene knockdown or knockout. In some embodiments SHMT1, SHMT2, MTHFD1, MTHFD1L, MTHFD2, MTHFD2L, SLC25A32, GPD1, GPD2, or PGP knockdown results in less than or equal to any of, 90, 80, 70, 60, 50, 45, 40, 35, 30, 25, 20, 15, 10, 5, 2.5 percent level of SHMT1, SHMT2, MTHFD1, MTHFD1L, MTHFD2, MTHFD2L, SLC25A32, GPD1, GPD2, or PGP expression or activity compared to unmodified cells.

Cells

Any cell susceptible to cell culture may be utilized in accordance with the present invention. In some embodiments, the cell is a mammalian cell. Non-limiting examples of mammalian cells that may be used in accordance with the present invention include BALB/c mouse myeloma line (NSO/I, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59,1977); baby hamster kidney cells (BHK, ATCC CCL 10); Chinese hamster ovary cells +/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2). In some preferred embodiment, the cells are CHO cells. In some preferred embodiments, the cells are GS-cells.

Additionally, any number of commercially and non-commercially available hybridoma cell lines may be utilized in accordance with the present invention. The term “hybridoma” as used herein refers to a cell or progeny of a cell resulting from fusion of an immortalized cell and an antibody-producing cell. Such a resulting hybridoma is an immortalized cell that produces antibodies. Individual cells used to create the hybridoma can be from any mammalian source, including, but not limited to, rat, pig, rabbit, sheep, pig, goat, and human. In some embodiments, a hybridoma is a trioma cell line, which results when progeny of heterohybrid myeloma fusions, which are the product of a fusion between human cells and a murine myeloma cell line, are subsequently fused with a plasma cell. In some embodiments, a hybridoma is any immortalized hybrid cell line that produces antibodies such as, for example, quadromas (See, e.g., Milstein et al., Nature, 537:3053, 1983). One skilled in the art will appreciate that hybridoma cell lines might have different nutrition requirements and/or might require different culture conditions for optimal growth, and will be able to modify conditions as needed.

Methods Comprising Identifying and/or Measuring the Metabolite Concentration in the Cell Culture Medium or in Cells (Cell Pellet Sample)

Metabolites can be identified and/or the concentration of metabolites (e.g. glycerol or formate) can be measured by any method known to the skilled person, including off line and on line measurement methods, such measurements also constitute metabolomic analysis or metabolomic measurement. Applied to the cell culture medium or to samples of the cells for instance the cell pellet

The identification and/or concentration of metabolites can be measured once or several times during the cell culture. In some embodiments, the metabolite concentration is measured continuously, intermittently, every 30 min, every hour, every two hours, twice a day, daily, or every two days. In a preferred embodiment the identification and/or concentration of metabolite is measured daily.

An off line measurement method as used herein refers to a method where the measurement of a parameter such as a concentration is not automated and integrated to the cell culture method. For example, a measurement method where a sample is manually taken from the cell culture medium so that a specific concentration can be measured in said sample is considered as an off line measurement method.

Online measurement methods as used herein refer to methods where the measurement of a parameter, such as a concentration, is automated and integrated to the cell culture method.

For example, a method using the Raman spectroscopy is an on-line measurement method. Alternatively, the use of High Performance Liquid Chromatography (HPLC) or Ultra Performance Liquid Chromatography (UPLC) based technology with an auto-sampler that draws samples from reactor and transfers them to the equipment in a programmed manner is an online measurement method.

The identification and/or concentration of metabolites can be measured by any method known to the skilled person. Preferred methods to identify and/or measure the concentration of metabolites in online or offline methods include for example Liquid Chromatography such as High-Performance Liquid Chromatography (HPLC), Ultra Performance Liquid Chromatography (UPLC) or Liquid Chromatography—Mass Spectrometry (LCMS), Nuclear Magnetic Resonance (NMR) or Gas Chromatography—Mass Spectrometry (GCMS).

In some embodiments, the identification and/or concentration of metabolite is measured off line by taking a sample of the cell culture medium and measuring the concentration of said at least one metabolite in said sample. In some embodiments, the identification and/or concentration of metabolite is measured on-line. In some embodiments, the identification and/or concentration of metabolite is measured online using Raman spectroscopy. In some embodiments, the identification and/or concentration of metabolite is measured online using HPLC or UPLC based technology with an auto-sampler that draws samples from reactor and transfers them to the equipment in a programmed manner. Identification of a metabolite includes determining the presence and/or identity of the metabolite.

Improvement of Cell Growth and Productivity

In some embodiments of the above described methods, cell growth and/or productivity are increased as compared to a control culture, said control culture being identical except that it does not comprise step the modified cells or the cells produced by the method of producing cells (i.e. the cells are unmodified.)

In some embodiments of the above described methods, the method of the invention is a method for improving cell growth. In some embodiments, the method of the invention is a method for improving cell growth in high density cell culture at high cell density.

High cell density as used herein refers to cell density above 1×10⁶cells/mL, 5×10⁶cells/mL, 1×10⁷cells/mL, 5×10⁷ cells/mL, 1×10⁸ cells/mL or 5×10⁸ cells/mL, preferably above 1×10⁷cells/mL, more preferably above 5×10⁷ cells/mL. In some embodiments, the above described methods are for improving cell growth in a cell culture where cell density is above 1×10⁶cells/mL, 5×10⁶cells/mL, 1×10⁷cells/mL, 5×10⁷ cells/mL, 1×10⁸ cells/mL or 5×10⁸ cells/mL . In some embodiments, the methods are for improving cell growth in a cell culture where maximum cell density is above 1×10⁶cells/mL, 5×10⁶cells/mL, 1×10⁷cells/mL, 5×10⁷ cells/mL, 1×10⁸ cells/mL or 5×10⁸ cells/mL.

In some embodiments, cell growth is determined by viable cell density (VCD), maximum viable cell density, or Integrated viable cell count (IVCC). In some embodiments, cell growth is determined by maximum viable cell density.

The term “viable cell density” as used herein refers to the number of cells present in a given volume of medium. Viable cell density can be measured by any method known to the skilled person. Preferably, Viable cell density is measured using an automated cell counter such as Bioprofile Flex®. The term maximum cell density as used herein refers to the maximum cell density achieved during the cell culture.

The term “cell viability” as used herein refers to the ability of cells in culture to survive under a given set of culture conditions or experimental variations. Those of ordinary skill in the art will appreciate that one of many methods for determining cell viability are encompassed in this invention. For example, one may use a dye (e.g., trypan blue) that does not pass through the membrane of a living cell, but can pass through the disrupted membrane of a dead or dying cell in order to determine cell viability.

The term “Integrated viable cell count (IVCC)” as used herein refers to as the area under the viable cell density (VCD) curve. IVCC can be calculated using the following formula: IVCCₜ₊₁=IVCCₜ₊(VCDₜ₊VCDₜ₊₁)*(Δt)/2 where Δt is the time difference between t and t+1 time points. IVCCₜ₌₀ can be assumed negligible. VCDₜ and VCDₜ₊₁ are viable cell densities at t and t+1 time points.

The term “titer” as used herein refers, for example, to the total amount of recombinantly expressed protein produced by a cell culture in a given amount of medium volume. Titer is typically expressed in units of grams of protein per liter of medium.

In some embodiments of the above described methods, cell growth is increased by at least 5%, 10%, 15%, 20% or 25% as compared to the control culture. In some embodiments, cell growth is increased by at least 10% as compared to the control culture. In some embodiments, cell growth is increased by at least 20% as compared to the control culture.

In some embodiments of the above described methods, the productivity is determined by titer and/or volumetric productivity.

The term “titer” as used herein refers, for example, to the total amount of recombinantly expressed protein produced by a cell culture in a given amount of medium volume. Titer is typically expressed in units of grams of protein per liter of medium.

In some embodiments of the above described methods, the productivity is determined by titer. In some embodiments, the productivity is increased by at least 5%, 10%, 15%, 20% or 25% as compared to the control culture. In some embodiments, the productivity is increased by at least 10% as compared to a control culture. In some embodiments, the productivity is increased by at least 20% as compared to a control culture.

In some embodiments of the above described methods, the maximum cell density of the cell culture is greater than 1×10⁶cells/mL, 5×10⁶cells/mL, 1×10⁷cells/mL, 5×10⁷ cells/mL, 1×10⁸ cells/mL or 5×10⁸ cells/mL. In some embodiments, the maximum cell density of the cell culture is greater than 5×10⁶cells/mL. In some embodiments, the maximum cell density of the cell culture is greater than 1×10⁸ cells/mL.

Cell Culture Media

The terms “medium”, “cell culture medium” and “culture medium” as used herein refer to a solution containing components or nutrients which nourish growing mammalian cells. Typically, the nutrients include essential and non-essential amino acids, vitamins, energy sources, lipids, and trace elements required by the cell for minimal growth and/or survival. Such a solution may also contain further nutrients or supplementary components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), inorganic compounds present at high final concentrations (e.g., iron), amino acids, lipids, and/or glucose or other energy source. In some embodiments, a medium is advantageously formulated to a pH and salt concentration optimal for cell survival and proliferation. In some embodiments, a medium is a feed medium that is added after the beginning of the cell culture.

A wide variety of mammalian growth media may be used in accordance with the present invention. In some embodiments, cells may be grown in one of a variety of chemically defined media, wherein the components of the media are both known and controlled. In some embodiments, cells may be grown in a complex medium, in which not all components of the medium are known and/or controlled.

Chemically defined growth media for mammalian cell culture have been extensively developed and published over the last several decades. All components of defined media are well characterized, and so defined media do not contain complex additives such as serum or hydrolysates. Early media formulations were developed to permit cell growth and maintenance of viability with little or no concern for protein production. More recently, media formulations have been developed with the express purpose of supporting highly productive recombinant protein producing cell cultures. Such media are preferred for use in the method of the invention. Such media generally comprises high amounts of nutrients and in particular of amino acids to support the growth and/or the maintenance of cells at high density. If necessary, these media can be modified by the skilled person for use in the method of the invention. For example, the skilled person may decrease the amount of phenylalanine, tyrosine, tryptophan and/or methionine in these media for their use as base media or feed media in a method as disclosed herein.

Not all components of complex media are well characterized, and so complex media may contain additives such as simple and/or complex carbon sources, simple and/or complex nitrogen sources, and serum, among other things. In some embodiments, complex media suitable for the present invention contains additives such as hydrolysates in addition to other components of defined medium as described herein.

In some embodiments, defined media typically includes roughly fifty chemical entities or components at known concentrations in water. Most of them also contain one or more well-characterized proteins such as insulin, IGF-1, transferrin or BSA, but others require no protein components and so are referred to as protein-free defined media. Typical chemical components of the media fall into five broad categories: amino acids, vitamins, inorganic salts, trace elements, and a miscellaneous category that defies neat categorization.

Cell culture medium may be optionally supplemented with supplementary components. The term “supplementary components” as used herein refers to components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In some embodiments, supplementary components may be added to the initial cell culture. In some embodiments, supplementary components may be added after the beginning of the cell culture.

Typically, components which are trace elements refer to a variety of inorganic salts included at micromolar or lower levels. For example, commonly included trace elements are zinc, selenium, copper, and others. In some embodiments, iron (ferrous or ferric salts) can be included as a trace element in the initial cell culture medium at micromolar concentrations. Manganese is also frequently included among the trace elements as a divalent cation (MnCl₂ or MnSO₄) in a range of nanomolar to micromolar concentrations. Numerous less common trace elements are usually added at nanomolar concentrations.

In some embodiments, the medium used in the methods of the invention is a medium suitable for supporting high cell density, such as for example 1×10⁶cells/mL, 5×10⁶cells/mL, 1×10⁷cells/mL, 5×10⁷ cells/mL, 1×10⁸ cells/mL or 5×10⁸ cells/mL, in a cell culture. In some embodiments, the cell culture is a mammalian cell fed-batch culture, preferably a CHO cells fed-batch culture.

Expression of Proteins

As noted above, in many instances the cells will be selected or engineered to produce high levels of desired products (e.g., recombinant protein or antibody). Often, cells will be manipulated by the hand of man to produce high levels of recombinant protein, for example by introduction of a gene encoding the protein of interest and/or by introduction of genetic control elements that regulate expression of that gene (whether endogenous or introduced).

Certain proteins may have detrimental effects on cell growth, cell viability or some other characteristic of the cells that ultimately limits production of the protein of interest in some way. Even amongst a population of cells of one particular type engineered to express a specific protein, variability within the cellular population exists such that certain individual cells will grow better, produce more protein of interest, or produce a protein with higher activity levels (e.g., enzymatic activity). In certain embodiments of the invention, a cell line is empirically selected by the practitioner for robust growth under the particular conditions chosen for culturing the cells. In some embodiments, individual cells engineered to express a particular protein are chosen for large-scale production based on cell growth, final cell density, percent cell viability, titer of the expressed protein or any combination of these or any other conditions deemed important by the practitioner. Any protein that is expressible in a host cell may be produced in accordance with the present teachings and may be produced according to the methods of the invention or by the cells of the invention. The term “host cell” as used herein refers to a cell that is manipulated according to the present invention to produce a protein of interest as described herein. A protein may be expressed from a gene that is endogenous to the cell, or from a heterologous gene that is introduced into the cell. A protein may be one that occurs in nature, or may alternatively have a sequence that was engineered or selected by the hand of man.

Proteins that may desirably be expressed in accordance with the present invention will often be selected on the basis of an interesting or useful biological or chemical activity. For example, the present invention may be employed to express any pharmaceutically or commercially relevant enzyme, receptor, antibody, hormone, regulatory factor, cytokine, antigen, binding agent, etc. In some embodiments, the protein expressed by cells in culture are selected from antibodies, or fragments thereof, nanobodies, single domain antibodies, glycoproteins, therapeutic proteins, growth factors, clotting factors, cytokines, fusion proteins, pharmaceutical drug substances, vaccines, enzymes, or Small Modular ImmunoPharmaceuticals™ (SMIPs). One of ordinary skill in the art will understand that any protein may be expressed in accordance with the present invention and will be able to select the particular protein to be produced based on his or her particular needs.

Antibodies

Given the large number of antibodies currently in use or under investigation as pharmaceutical or other commercial agents, production of antibodies is of particular interest in accordance with the present invention. Antibodies are proteins that have the ability to specifically bind a particular antigen. Any antibody that can be expressed in a host cell may be produced in accordance with the present invention and may be produced according to the methods of the invention or by the cells of the invention. In some embodiments, the antibody to be expressed is a monoclonal antibody.

In some embodiments, the monoclonal antibody is a chimeric antibody. A chimeric antibody contains amino acid fragments that are derived from more than one organism. Chimeric antibody molecules can include, for example, an antigen binding domain from an antibody of a mouse, rat, or other species, with human constant regions. A variety of approaches for making chimeric antibodies have been described. See e.g., Morrison et al., Proc. Natl. Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

In some embodiments, the monoclonal antibody is a human antibody derived, e.g., through the use of ribosome-display or phage-display libraries (see, e.g., Winter et al., U.S. Pat. No. 6,291,159 and Kawasaki, U.S. Pat. No. 5,658,754) or the use of xenographic species in which the native antibody genes are inactivated and functionally replaced with human antibody genes, while leaving intact the other components of the native immune system (see, e.g., Kucherlapati et al., U.S. Pat. No. 6,657,103).

In some embodiments, the monoclonal antibody is a humanized antibody. A humanized antibody is a chimeric antibody wherein the large majority of the amino acid residues are derived from human antibodies, thus minimizing any potential immune reaction when delivered to a human subject. In humanized antibodies, amino acid residues in the complementarity determining regions are replaced, at least in part, with residues from a non-human species that confer a desired antigen specificity or affinity. Such altered immunoglobulin molecules can be made by any of several techniques known in the art, (e.g., Teng et aL, Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and are preferably made according to the teachings of PCT Publication WO92/06193 or EP 0239400, all of which are incorporated herein by reference). Humanized antibodies can be commercially produced by, for example, Scotgen Limited, 2 Holly Road, Twickenham, Middlesex, Great Britain. For further reference, see Jones et al., Nature 321:522-525 (1986); Riechmann et al., Nature 332:323-329 (1988); and Presta, Curr. Op. Struct. Biol. 2:593-596 (1992), all of which are incorporated herein by reference.

In some embodiments, the monoclonal, chimeric, or humanized antibodies described above may contain amino acid residues that do not naturally occur in any antibody in any species in nature. These foreign residues can be utilized, for example, to confer novel or modified specificity, affinity or effector function on the monoclonal, chimeric or humanized antibody. In some embodiments, the antibodies described above may be conjugated to drugs for systemic pharmacotherapy, such as toxins, low-molecular-weight cytotoxic drugs, biological response modifiers, and radionuclides (see e.g., Kunz et al., Calicheamicin derivative-carrier conjugates, US20040082764 A1).

In general, practitioners of the present invention will select a protein of interest, and will know its precise amino acid sequence. Any given protein that is to be expressed in accordance with the present invention may have its own particular characteristics and may influence the cell density or viability of the cultured cells, may be expressed at lower levels than another protein grown under identical culture conditions, and may have different biological activity depending on the exact culture conditions and steps performed. One of ordinary skill in the art will be able to appropriately modify the steps and compositions used to produce a particular protein according to the teachings of the present invention in order to optimize cell growth and the production and/or activity level of any given expressed protein.

Introduction of Genes for the Expression of Proteins into Host Cells

Generally, a nucleic acid molecule introduced into the cell encodes the protein desired to be expressed according to the present invention and may be introduced and expressed according to the methods of the invention or into and by the cells of the invention. Alternatively, a nucleic acid molecule may encode a gene product that induces the expression of the desired protein by the cell. For example, introduced genetic material may encode a transcription factor that activates transcription of an endogenous or heterologous protein. Alternatively or additionally, an introduced nucleic acid molecule may increase the translation or stability of a protein expressed by the cell.

Methods suitable for introducing nucleic acids sufficient to achieve expression of a protein of interest into mammalian host cells are known in the art. See, for example, Gething et al., Nature, 293:620-625, 1981; Mantei et al., Nature, 281:40-46, 1979; Levinson et al. EP 117,060; and EP 117,058, each of which is incorporated herein by reference. For mammalian cells, common methods of introducing genetic material into mammalian cells include the calcium phosphate precipitation method of Graham and van der Erb (Virology, 52:456-457, 1978) or the lipofectamine™ (Gibco BRL) Method of Hawley-Nelson (Focus 15:73, 1993). General aspects of mammalian cell host system transformations have been described by Axel in U.S. Pat. No. 4,399,216 issued Aug. 16, 1983. For various techniques for introducing genetic material into mammalian cells, see Keown et al., Methods in Enzymology, 1989, Keown et al., Methods in Enzymology, 185:527-537, 1990, and Mansour et al., Nature, 336:348-352, 1988. Additional methods suitable for introducing nucleic acids include electroporation, for example as employed using the GenePulser XCell™ electroporator by BioRad™.

In some embodiments, a nucleic acid to be introduced is in the form of a naked nucleic acid molecule. For example, the nucleic acid molecule introduced into a cell may consist only of the nucleic acid encoding the protein and the necessary genetic control elements. Alternatively, a nucleic acid encoding the protein (including the necessary regulatory elements) may be contained within a plasmid vector. Non-limiting representative examples of suitable vectors for expression of proteins in mammalian cells include pCDNA1; pCD, see Okayama, et al. Mol. Cell Biol. 5:1136-1142, 1985; pMClneo Poly-A, see Thomas, et al. Cell 51:503-512, 1987; a baculovirus vector such as pAC 373 or pAC 610; CDM8 , see Seed, B. Nature 329:840, 1987; and pMT2PC, see Kaufman, et al. EMBO J. 6:187-195, 1987, each of which is incorporated herein by reference in its entirety. In some embodiments, a nucleic acid molecule to be introduced into a cell is contained within a viral vector. For example, a nucleic acid encoding the protein may be inserted into the viral genome (or a partial viral genome). Regulatory elements directing the expression of the protein may be included with the nucleic acid inserted into the viral genome (i.e., linked to the gene inserted into the viral genome) or can be provided by the viral genome itself.

Naked DNA can be introduced into cells by forming a precipitate containing the DNA and calcium phosphate. Alternatively, naked DNA can also be introduced into cells by forming a mixture of the DNA and DEAE-dextran and incubating the mixture with the cells or by incubating the cells and the DNA together in an appropriate buffer and subjecting the cells to a high-voltage electric pulse (e.g., by electroporation). A further method for introducing naked DNA cells is by mixing the DNA with a liposome suspension containing cationic lipids. The DNA/liposome complex is then incubated with cells. Naked DNA can also be directly injected into cells by, for example, microinjection.

Alternatively, naked DNA can also be introduced into cells by complexing the DNA to a cation, such as polylysine, which is coupled to a ligand for a cell-surface receptor (see for example Wu, G. and Wu, C.H. J. Biol. Chem. 263:14621, 1988; Wilson et al. J. Biol. Chem. 267:963-967, 1992; and U.S. Pat. No. 5,166,320, each of which is hereby incorporated by reference in its entirety). Binding of the DNA-ligand complex to the receptor facilitates uptake of the DNA by receptor-mediated endocytosis.

Use of viral vectors containing particular nucleic acid sequences, e.g., a cDNA encoding a protein, is a common approach for introducing nucleic acid sequences into a cell. Infection of cells with a viral vector has the advantage that a large proportion of cells receive the nucleic acid, which can obviate the need for selection of cells which have received the nucleic acid. Additionally, molecules encoded within the viral vector, e.g., by a cDNA contained in the viral vector, are generally expressed efficiently in cells that have taken up viral vector nucleic acid. Defective retroviruses are well characterized for use in gene transfer for gene therapy purposes (for a review see Miller, A.D. Blood 76:271, 1990). A recombinant retrovirus can be constructed having a nucleic acid encoding a protein of interest inserted into the retroviral genome. Additionally, portions of the retroviral genome can be removed to render the retrovirus replication defective. Such a replication defective retrovirus is then packaged into virions which can be used to infect a target cell through the use of a helper virus by standard techniques.

The genome of an adenovirus can be manipulated such that it encodes and expresses a protein of interest but is inactivated in terms of its ability to replicate in a normal lytic viral life cycle. See, for example, Berkner et al. BioTechniques 6:616, 1988; Rosenfeld et al. Science 252:431-434, 1991; and Rosenfeld et al. Cell 68:143-155, 1992. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses are advantageous in that they do not require dividing cells to be effective gene delivery vehicles and can be used to infect a wide variety of cell types, including airway epithelium (Rosenfeld et al., 1992, cited supra), endothelial cells (Lemarchand et al., Proc. Natl. Acad. Sci. USA 89:6482-6486, 1992), hepatocytes (Herz and Gerard, Proc. Natl. Acad. Sci. USA 90:2812-2816, 1993) and muscle cells (Quantin et al., Proc. Natl. Acad. Sci. USA 89:2581-2584, 1992). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham, J. Virol. 57:267, 1986). Most replication-defective adenoviral vectors currently in use are deleted for all or parts of the viral El and E3 genes but retain as much as 80% of the adenoviral genetic material. Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol., 158:97-129, 1992). It is also one of the few viruses that may integrate its DNA into non-dividing cells, and exhibits a high frequency of stable integration (see for example Flotte et al., Am. J. Respir. Cell. Mol. Biol. 7:349-356, 1992; Samulski et al., J. Virol. 63:3822-3828, 1989; and McLaughlin et al., J. Virol. 62:1963-1973, 1989). Vectors containing as little as 300 base pairs of AAV can be packaged and can integrate. Space for exogenous DNA is limited to about 4.5 kb. An AAV vector such as that described in Tratschin et al. (Mol. Cell. Biol. 5:3251-3260, 1985) can be used to introduce DNA into cells. A variety of nucleic acids have been introduced into different cell types using AAV vectors (see for example Hermonat et al., Proc. Natl. Acad. Sci. USA 81:6466-6470, 1984; Tratschin et al., Mol. Cell. Biol. 4:2072-2081, 1985; Wondisford et al., Mol. Endocrinol. 2:32-39, 1988; Tratschin et al., J. Virol. 51:611-619, 1984; and Flotte et al., J. Biol. Chem. 268:3781-3790, 1993).

When the method used to introduce nucleic acid molecules into a population of cells results in modification of a large proportion of the cells and efficient expression of the protein by the cells, the modified population of cells may be used without further isolation or subcloning of individual cells within the population. That is, there may be sufficient production of the protein by the population of cells such that no further cell isolation is needed and the population can be immediately be used to seed a cell culture for the production of the protein. Alternatively, it may be desirable to isolate and expand a homogenous population of cells from a few cells or a single cell that efficiently produce(s) the protein. Alternative to introducing a nucleic acid molecule into a cell that encodes a protein of interest, the introduced nucleic acid may encode another polypeptide or protein that induces or increases the level of expression of the protein produced endogenously by a cell. For example, a cell may be capable of expressing a particular protein but may fail to do so without additional treatment of the cell. Similarly, the cell may express insufficient amounts of the protein for the desired purpose. Thus, an agent that stimulates expression of the protein of interest can be used to induce or increase expression of that protein by the cell. For example, the introduced nucleic acid molecule may encode a transcription factor that activates or upregulates transcription of the protein of interest. Expression of such a transcription factor in turn leads to expression, or more robust expression of the protein of interest.

In certain embodiments, a nucleic acid that directs expression of the protein is stably introduced into the host cell. In certain embodiments, a nucleic acid that directs expression of the protein is transiently introduced into the host cell. One of ordinary skill in the art will be able to choose whether to stably or transiently introduce a nucleic acid into the cell based on his or her experimental needs. A gene encoding a protein of interest may optionally be linked to one or more regulatory genetic control elements. In certain embodiments, a genetic control element directs constitutive expression of the protein. In certain embodiments, a genetic control element that provides inducible expression of a gene encoding the protein of interest can be used. The use of an inducible genetic control element (e.g., an inducible promoter) allows for modulation of the production of the protein in the cell. Non-limiting examples of potentially useful inducible genetic control elements for use in eukaryotic cells include hormone-regulated elements (e.g., see Mader, S. and White, J. H., Proc. Natl. Acad. Sci. USA 90:5603-5607, 1993), synthetic ligand-regulated elements (see, e.g. Spencer, D. M. et al., Science 262:1019-1024, 1993) and ionizing radiation-regulated elements (e.g., see Manome, Y. et al., Biochemistry 32:10607-10613, 1993; Datta, R. et al., Proc. Natl. Acad. Sci. USA 89:10149-10153, 1992). Additional cell-specific or other regulatory systems known in the art may be used in accordance with the invention.

One of ordinary skill in the art will be able to choose and, optionally, to appropriately modify the method of introducing genes that cause the cell to express the protein of interest in accordance with the teachings of the present invention.

Isolation of the Expressed Protein

In general, it will typically be desirable to isolate and/or purify proteins expressed according to the present invention. In certain embodiments, the expressed protein is secreted into the medium and thus cells and other solids may be removed, as by centrifugation or filtering for example, as a first step in the purification process.

Alternatively, the expressed protein may be bound to the surface of the host cell. For example, the media may be removed and the host cells expressing the protein are lysed as a first step in the purification process. Lysis of mammalian host cells can be achieved by any number of means well known to those of ordinary skill in the art, including physical disruption by glass beads and exposure to high pH conditions.

The expressed protein may be isolated and purified by standard methods including, but not limited to, chromatography (e.g., ion exchange, affinity, size exclusion, and hydroxyapatite chromatography), gel filtration, centrifugation, or differential solubility, ethanol precipitation and/or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression : A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol. 182), Academic Press, 1997, each of which is incorporated herein by reference). For immunoaffinity chromatography in particular, the protein may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the protein during the purification process. Protease inhibitors are particularly advantageous when cells must be lysed in order to isolate and purify the expressed protein.

One of ordinary skill in the art will appreciate that the exact purification technique will vary depending on the character of the protein to be purified, the character of the cells from which the protein is expressed, and/or the composition of the medium in which the cells were grown.

Pharmaceutical Formulations

In certain preferred embodiments of the invention, produced polypeptides or proteins will have pharmacologic activity and will be useful in the preparation of pharmaceuticals. Inventive compositions as described above may be administered to a subject or may first be formulated for delivery by any available route including, but not limited to parenteral (e.g., intravenous), intradermal, subcutaneous, oral, nasal, bronchial, ophthalmic, transdermal (topical), transmucosal, rectal, and vaginal routes. Inventive pharmaceutical compositions typically include a purified polypeptide or protein expressed from a mammalian cell line, a delivery agent (i.e., a cationic polymer, peptide molecular transporter, surfactant, etc., as described above) in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

The polypeptide or protein expressed according to the present invention can be administered at various intervals and over different periods of time as required, e.g., one time per week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with a polypeptide or protein as described herein can include a single treatment or, in many cases, can include a series of treatments. It is furthermore understood that appropriate doses may depend upon the potency of the polypeptide or protein and may optionally be tailored to the particular recipient, for example, through administration of increasing doses until a preselected desired response is achieved. It is understood that the specific dose level for any particular animal subject may depend upon a variety of factors including the activity of the specific polypeptide or protein employed, the age, body weight, general health, gender, and diet of the subject, the time of administration, the route of administration, the rate of excretion, any drug combination, and the degree of expression or activity to be modulated.

The present invention includes the use of compositions for treatment of nonhuman animals. Accordingly, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001. Pharmaceutical compositions can be included in a container, pack, or dispenser together with instructions for administration.

In some aspects, any embodiment provided herein may be used in combination with a method, cell, or other embodiment provided in WO2017051347 (“Cells and Method of Cell Culture”) and WO2015140708 (“Method of Cell Culture”), both of which are hereby incorporated by reference for all purposes.

Incorporated by reference herein for all purposes is the content of U.S. Provisional Patent Application Nos. 62/776,190 (filed Dec. 6, 2018) and 62/932,096 (Filed Nov. 7, 2019).

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims. The contents of all figures and all references, patents and published patent applications cited throughout this application are expressly incorporated herein by reference.

### EXAMPLES

### Example 1

Formate and glycerol as Metabolic Byproducts Accumulating in Fedbatch (HiPDOG) Cultures of CHO Cells

Goal

This experiment was carried out to identify the levels to which formate and glycerol accumulate in the glucose-restricted and conventional fed-batch cultures of mammalian cells.

Materials and Methods

Cells and Medium

CHO cells utilizing a glutamine synthase expression system and expressing a recombinant antibody were used in the current experiment. Two types of medium were used in this experiment. First medium is “Medium A” which is used for inoculation of the experiment on day 0 of the culture. Second medium is “Medium B” which is the enriched nutrient media used as a feed media for conventional and HIPDOG fed-batch processes (described in the next section).

Medium A is a fortified version of insulin-free Medium 9 (U.S. Pat. No. 7,294,484, table 14), with slight differences in concentrations of sodium bicarbonate and potassium chloride, and containing Pluronic F68 instead of polyvinyl alcohol. It was fortified by adding 10% glutamine-free Medium 5 (U.S. Pat. No. 7,294,484, table 7), and by further raising the concentrations of eight amino acids (Glu, Tyr, Gly, Phe, Pro, Thr, Trp and Val). The concentrations of amino acids are listed in the Table 1 below.

TABLE 1
Concentration of Amino Acids in Medium A

| Amino Acids | Concentration in Medium A (mM) |
|---|---|
| alanine | 0.4 |
| arginine | 5.3 |
| asparagine•H2O | 21.1 |
| aspartic acid | 2.3 |
| cysteine•HCl•H2O | 0.4 |
| cystine•2HCl | 1.5 |
| glutamic acid | 0 |
| monosodium glutamate | 2.0 |
| glutamine | 0 |
| glycine | 3.6 |
| histidine•HCl•H2O | 2.7 |
| isoleucine | 5.4 |
| leucine | 9.4 |
| lysine•HCl | 8.9 |
| methionine | 3.1 |
| phenylalanine | 4.5 |
| proline | 9.1 |
| serine | 11.8 |
| threonine | 10.8 |
| tryptophan | 2.3 |
| tyrosine•2Na•2H2O | 5.1 |
| valine | 10.3 |

Medium B has the same composition as Medium 5 (U.S. Pat. No. 7,294,484, table 7), but with higher levels of the amino acids (by a factor of 2.5). The concentrations of amino acids in Medium B are shown in Table 2.

TABLE 2
Concentration of Amino Acids in Medium B

| Amino Acids | Concnetration in Medium B (mM) |
|---|---|
| alanine | 6.0 |
| arginine | 32.9 |
| asparagine•H₂O | 54.0 |
| aspartic acid | 15.0 |
| cysteine•HCl•H₂O | 0.0 |
| cystine•2HCl | 4.7 |
| glutamic acid | 6.0 |
| monosodium glutamate | 0.0 |
| glutamine | 0.0 |
| glycine | 6.0 |
| histidine•HCl•H₂O | 10.5 |
| isoleucine | 27.0 |
| leucine | 38.9 |
| lysine•HCl | 30.0 |
| methionine | 12.0 |
| phenylalanine | 15.0 |
| proline | 18.0 |
| serine | 45.2 |
| threonine | 24.0 |
| tryptophan | 4.8 |
| tyrosine•2Na•2H₂O | 12.0 |
| valine | 24.0 |

Bioreactor Setup

Two conditions were employed, a conventional fed-batch process and glucose-restricted fed-batch process. In the glucose-restricted fed-batch process (hereafter HIPDOG culture), glucose was limited by using the HIPDOG process (Gagnon et al, 2011). The pH dead-band used while the HIPDOG control was operational was 7.025+/−0.025.

The conventional process was identical to the HIPDOG process with respect to inoculum cell density targeted (1 E6 cells/mL), the media used, culture volume (1.1 L), the amount of feed added daily to the culture, and the process parameters including the temperature (36.5 C), pH (6.9-7.2) and the agitation rate (267 rpm). The only difference was in the glucose level : in the conventional culture, it was maintained at greater than 2 g/L between days 2 through 5; in the HIPDOG culture glucose was consumed by the cells naturally until the glucose level fell to a point at which the cells began to also consume lactic acid (observed by a slight rise in pH of the culture) and the HIPDOG technology/feeding strategy commenced. Post day 5 the glucose levels in both the conditions were maintained at concentrations more than 2 g/L by feeding glucose as necessary. Also, post day 5 both the cultures were treated similarly until day 12. Viable cell density, lactate and ammonia concentration in the cell culture medium were measured on a daily basis for both the conditions. The base media used is Media A and the feed media used was Media B.

For culture metabolite analysis, spent media samples and the cell pellet samples were collected and analyzed from duplicate reactors runs, performed for each condition. Time points considered for the analysis include days 0, 2, 3, 5, 7, 9 and 10. NMR technique was employed to assess the concentrations to which formate and glycerol accumulate at different time points of the two cultures. The details of the sample preparation and the NMR method employed are described below.

NMR Sample Preparation, Data Acquisition and Processing

1000 μL of each sample was filtered using Nanosep 3K Omega microcentrifuge filter tubes for 60 minutes, and 630 μL of the filtered sample was used for NMR analysis. These filters are preserved with glycerol, and as such some trace amounts of glycerol may appear in the analysis. Internal standard solution was added to each sample solution, and the resulting mixture was vortexed for 30s. 700 μL of the centrifuged solution was transferred to an NMR tube for data acquisition.

NMR spectra were acquired on a Varian four-channel VNMRS 700 MHz NMR spectrometer equipped with a cryogenically cooled 1H/13C triple resonance biomolecular probe with auto tuning. The pulse sequence used was a 1 D-tnnoesy with a 990 ms presaturation on water and a 4 s acquisition time. Spectra were collected with 32 transients and 4 steady-state scans at 298 K.

Spectra were processed and .cnx files were generated using the Processor module in Chenomx NMR Suite 8.0. Compounds were identified and quantified using the Profiler module in Chenomx NMR Suite 8.0 with the Chenomx Compound Library version 9, containing 332 compounds. For reporting purposes, the profiled concentrations have been corrected to reflect the composition of the original sample, instead of the contents of the NMR tube. During sample preparation, each sample is diluted by introducing an internal standard and, where necessary, to increase the analyzed volume of a small sample.

Results

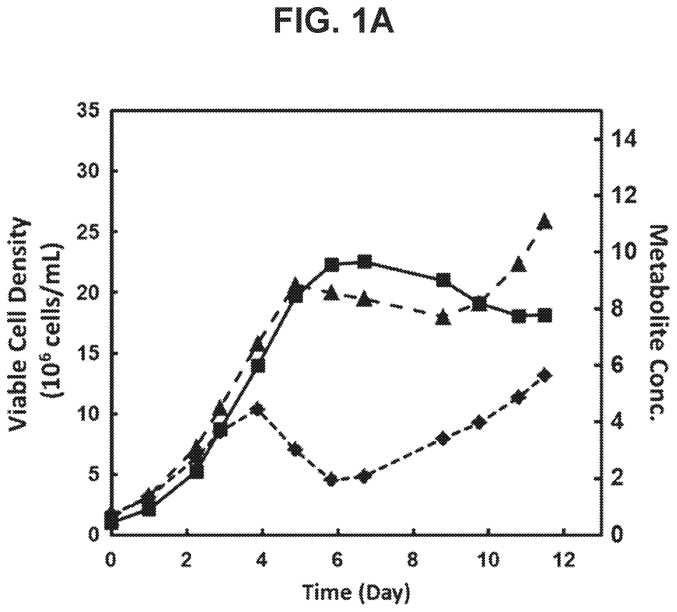

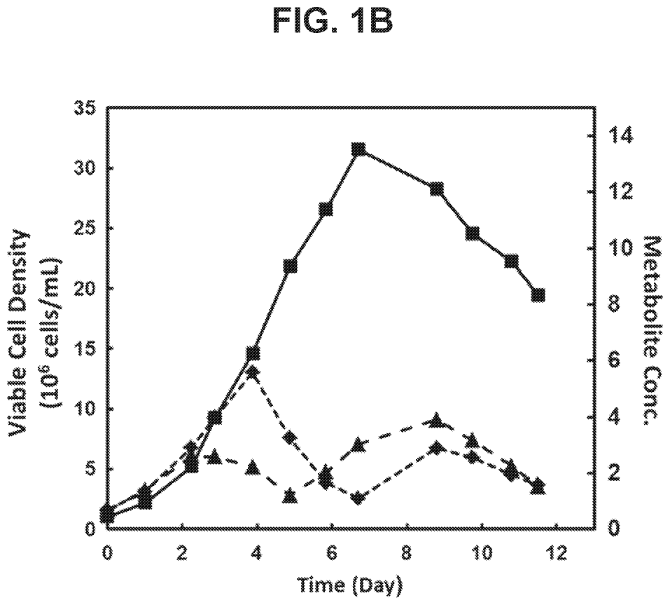

Initially cells grew exponentially in both conventional and HIPDOG cultures and attained peak cells densities on day 6 and day 7, respectively, with HIPDOG culture peaking at much higher cell densities (FIGS. 1A and 1B). The lactate levels in the HIPDOG process remained low due to application of the HIPDOG control (between day 2-day 5) whereas the lactate levels accumulated to very high levels in case of the conventional fed-batch culture. Ammonia was also maintained at low levels during the conventional and HIPDOG culture by the use of cells comprising a glutamine synthetase expression system. The titer (amount of protein of interest per liter of cell culture medium) was measured at the end of the culture (Day 12). For the conventional process, the titer at Day 12 was 2.3 g/L ; for the HIPDOG process, the titer at Day 12 was 4 g/L. The HIPDOG culture attained higher titer compared to the conventional process. The differences in the cell densities and titer values are likely an outcome of the differences in the lactate accumulations observed between the two cultures.

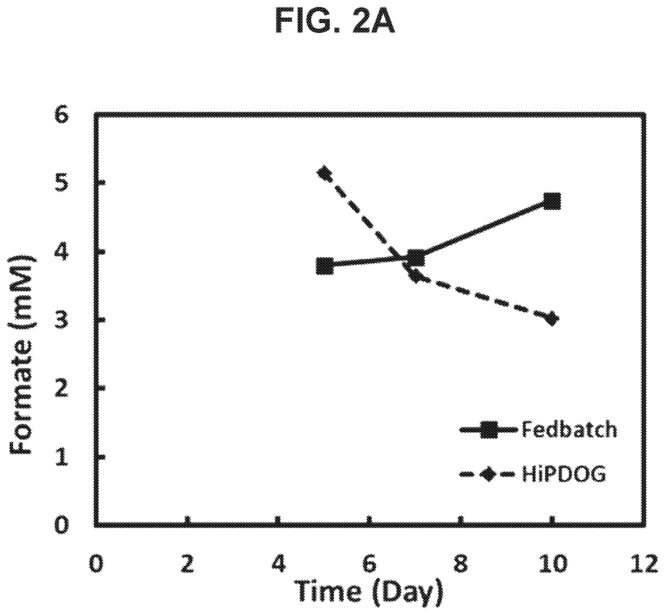

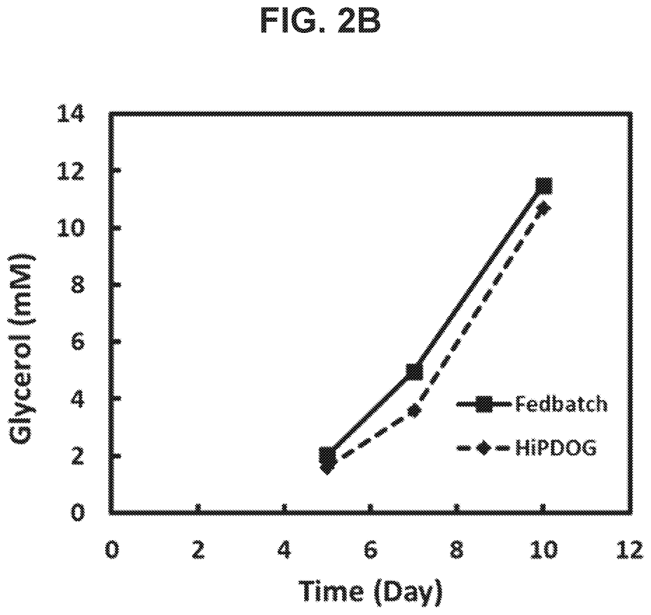

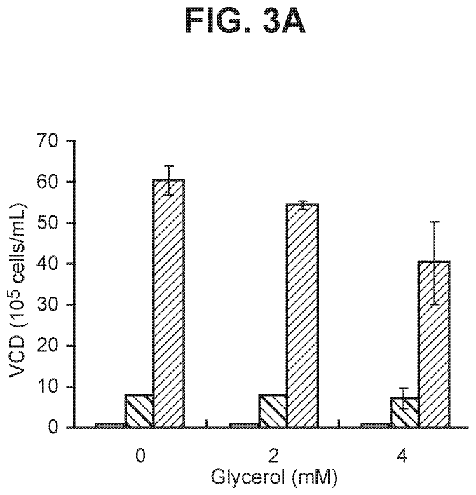

Formate and glycerol level in the culture were quantified using NMR approach. FIGS. 2A and 2B show the time course concentration profiles of formate and glycerol in HiPDOG and conventional fedbatch cultures. In both the conditions, formate accumulates to 4-5 mM range by day 5. Post day 5, the concentration profiles are slightly different between the two conditions. Glycerol, on the other hand, accumulates in both HiPDOG and conventional fedbatch conditions over the course of the culture and reaches concentrations above 10 mM by day 10 of both the cultures. Formate and glycerol are known to be byproducts of serine catabolism and glycolysis pathway, respectively. Since serine can be biosynthesized from glycine and threonine, even glycine and threonine can contribute towards formate production.

### Example 2

Probing the Effect of formate and glycerol on Proliferative Capability of CHO Cells in Culture

Goal

Formate and glycerol are byproducts of serine catabolism and glycolysis pathways, respectively, which were observed to accumulate in the HiPDOG and conventional fed-batch cultures of CHO cells. In these cultures, the peak level of accumulation observed for formate and glycerol were 5 mM and 10 mM, respectively. This experiment was setup to probe the effect of these two compounds, individually, on growth of CHO cells within or below the concentration range observed in the HiPDOG or conventional fed-batch cultures.

Materials and Methods

CHO cells (cell line A or cell line B) producing a recombinant antibody were inoculated at low cell densities (0.1 E6 cells/mL) in various conditions in triplicates in 6-well plate cultures. The working volume for each well on day 0 was 4 mL. Cell line A was inoculated either in fresh Medium A or fresh Medium A supplemented with formate at various concentrations. Cell line B was inoculated either in fresh Medium A or fresh Medium A supplemented with glycerol at various concentrations. The concentrations tested for formate were [0, 2, 4 and 6 mM] and for glycerol were [0, 2 and 4 mM]. The 6-well plates were incubated on a shaking platform in 36.5C and 5% carbon dioxide. Cell growth in all the conditions was monitored for 5 or 6 days.

Results

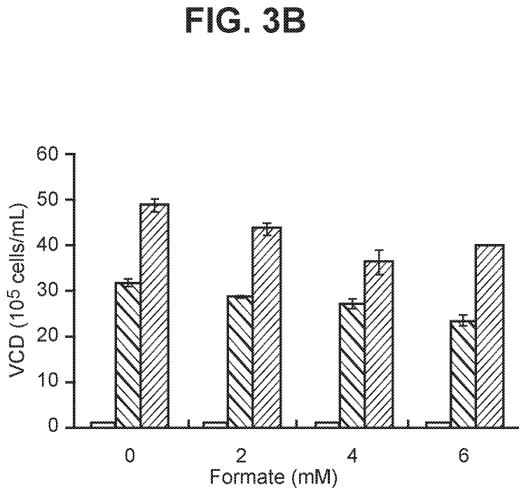

FIGS. 3A and 3B show the independent effect of the formate or glycerol on growth of the CHO cells. Cells cultured in fresh medium grew very well. Cell growth was suppressed when cells were cultured in fresh media supplemented with glycerol (FIG. 3A) or formate (FIG. 3B) at concentrations higher than 2 mM. This demonstrates that formate and glycerol inhibit cell growth, independently. Formate is a metabolic byproduct of serine (or glycine or threonine) catabolism and glycerol is a metabolic byproduct of glucose metabolism (glycolysis).

### Example 3

Assessing the Accumulation of formate Through Limitation of serine supplementation in Fed-Batch Cultures of CHO Cells.

Goal

The main goal of this example was to assess reduction in the accumulation of formate in HiPDOG cultures of CHO cells by limiting the supply of the serine.

Materials and Methods

Cells and Bioreactor Setup

CHO cells (cell line C) expressing a recombinant antibody were used in this example. Two conditions were tested as part of this example: A) HiPDOG culture with low levels of 10 amino acids including serine (Low AA), B) HiPDOG culture with normal amino acids concentrations (Control). The experiment was carried out for 12 days.

Exponentially growing cells from seed cultures were inoculated at about 1×10⁶ cells/mL into production bioreactors that employed a typical fed-batch process (with typical levels of amino acids) or the low amino acid fed-batch process. In the low amino acid condition, the concentrations of 10 amino acids including leucine, isoleucine, valine, methionine, serine, threonine, glycine and tyrosine were maintained around 0.5 mM for first seven days of the culture after which they were allowed to increase beyond 1 mM. Viable cell density, glucose, lactate, ammonia and amino acid concentrations were measured on daily basis until day 12. For both the conditions, the culture volume (1L) and the process parameters including the temperature (36.5 C), pH and the agitation rate (259 rpm) were identical. pH during HiPDOG phase of the culture (day 0-day 7) was 7.025+/−0.025 and post HiPDOG phase (day 7-day 12) was 7.05+/−0.15. The base media used in control condition was Medium A and that used in low amino acid conditions was the modified version of Medium A with low concentrations of amino acids, including tyrosine, phenylalanine, methionine, tryptophan, serine, glycine, threonine, leucine, isoleucine and valine. The feed medium used for both the conditions was modified version of Medium B described in example. Amino acid levels in modified version of Medium B were adjusted such that the amount of amino acid delivered through feeding this Medium B, in a semi-continuous fashion, is approximately equal to the amount of amino acid taken up by the culture. The feed rate used was proportional to integral viable cells in the culture. Supernatant samples from both the conditions were analyzed for the levels of the formate using the NMR technology described in Example 1. Culture amino acid levels were measured using amino acid analysis method.

Amino Acid Analysis

10 μL of either a standard amino acid mix solution or a spent medium sample (10 times diluted sample) was mixed with 70 μL of AccQTag Ultra borate buffer (Waters UPLC AAA H-Class Applications Kit [176002983]), and 20 pL of AccQTag reagent previously dissolved in 1.0 mL of AccQ.Tag Ultra reagent diluent was added. The reaction was allowed to proceed for 10 min at 55° C. Liquid chromatographic analysis was performed on a Waters Acquity UPLC system, equipped with a binary solvent manager, an autosampler, a column heater and a PDA detector. The separation column was a Waters AccQTag Ultra column (2.1 mm i.d. ×100 mm, 1.7 μηl particles). The column heater was set at 55° C., and the mobile phase flow rate was maintained at 0.7 mL/min. Eluent A was 10% AccQTag Ultra concentrate solvent A, and eluent B was 100% AccQTag Ultra solvent B. The nonlinear separation gradient was 0-0.54 min (99.9% A), 5.74 min (90.0% A), 7.74 min (78.8% A), 8.04-8.64 min (40.4% A), 8.73-10 min (99.9% A). One microliter of sample was injected for analysis. The PDA detector was set at 260 nm. The previously determined elution times for the amino acids are used to identify the specific amino acid peaks on the chromatogram for each sample. The amino acid concentrations were estimated using the area under the peak and the calibration curve generated using the standard solution (Amino Acids Standard H, Thermo Scientific, PI-20088).

Results

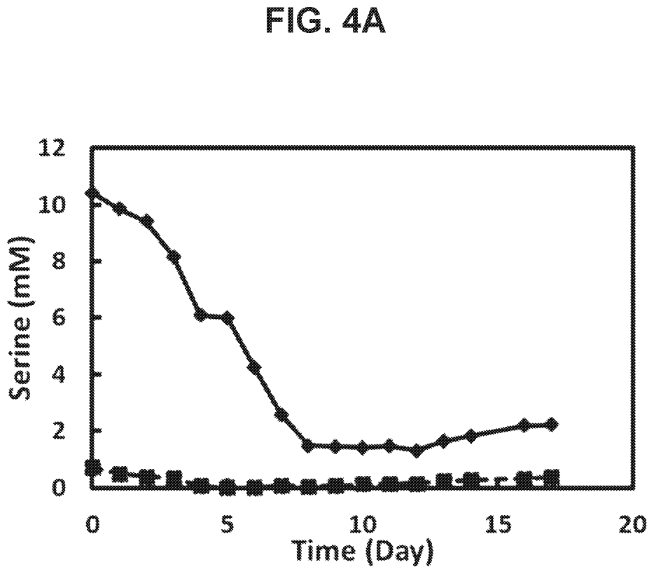

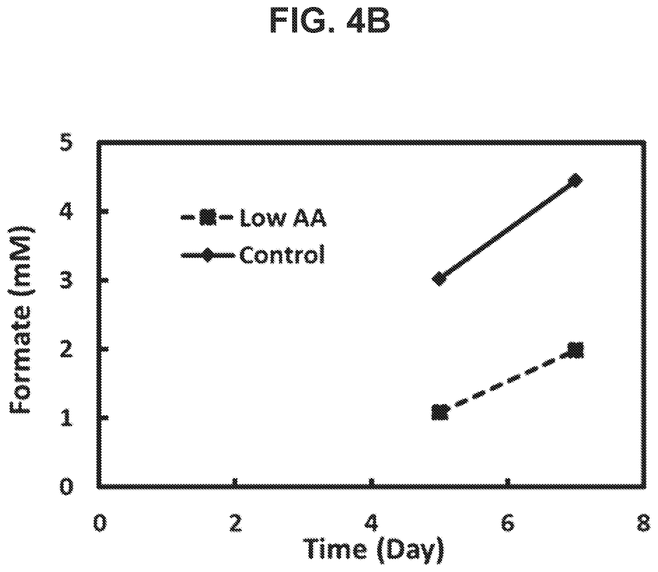

The concentrations of the serine were successfully maintained between around or below 0.5 mM in the Low AA condition (FIG. 4A). Such limitation of serine levels in the Low AA condition resulted in lower accumulation of formate (FIG. 4B).

### Example 4

Experiment to Determine the Gene Targets for Metabolically Engineering CHO Cells Towards Suppression of formate Production.

Goal

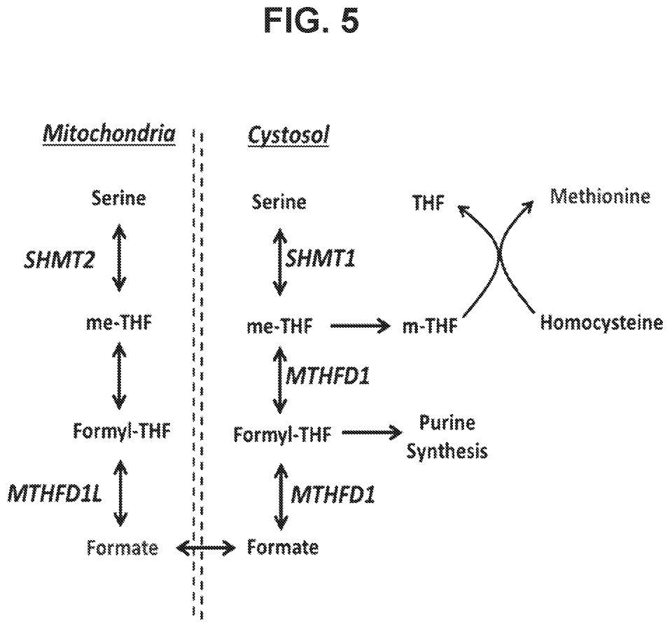

The cause for biosynthesis of the growth inhibitor formate in CHO cells was examined. Formate is a byproduct of serine catabolism, which is known to take place in the mitochondrion as well as in the cytosol of mammalian cells (FIG. 5). Expression (mRNA) levels of genes encoding relevant catabolic enzymes in the mitochondrial and cytosolic catabolic pathway of serine were probed using RNAseq analysis. Based on the gene expression data, targets for metabolic engineering were identified in an effort to suppress the serine flux towards the production of formate.

Materials and Methods

A Pfizer proprietary RNAseq database was queried to provide a baseline expectation of gene expression levels, and by extension, the likely enzymatic activity of the key enzymes involved in serine catabolism. The RNAseq data was derived from CHO cell line D which shares the same host as cell line C. Enzymes analyzed include SHMT1, SHMT2, MTHFD1, MTHFD1L, MTHFD2, MTHFD2L, SLC25A32. RNAseq data for B-actin was included for reference. Data was reported as reads per kilobase of transcript per million mapped reads (RPKM).

Results

The gene expression levels of the enzymes involved in serine catabolism resulting in formate biosynthesis are shown in Table 3. Enzymes in both mitochondrial and cytosolic catabolic pathways of serine are expressed. However, the first enzyme in the mitochondrial catabolic pathway, SHMT2, is expressed at a higher level than corresponding cytosolic catabolic enzyme, SHMT1. This suggests that mitochondrial pathway might be more active. Since, SHMT2 is first enzyme, suppressing the activity of SHMT2 enzyme genetically can significantly reduce formate production.

TABLE 3
RNAseq data for genes involved in mitochondrial and
cytosolic serine catabolic pathways. The data are
reported in reads per kilobase million (RPKM).

| Gene | RPKM |
|---|---|
| SHMT1 | 11 |
| SHMT2 | 55 |
| MTHFD1 | 34 |
| MTHFD1L | 9 |
| MTHFD2 | 68 |
| MTHFD2L | 3 |
| SLC25A32 | 5 |
| ACTB | 506 |

### Example 5

Down-Regulation of SHMT2 Expression Levels in CHO Cells to Suppress the Production of formate

The expression of SHMT2 is targeted for down-regulation using molecular biology techniques that reduce gene expression (knock-down) or render the gene inoperative (knock-out). Techniques that knock-down gene expression, often referred to as RNA interference (RNAi), include micro RNA (miRNA), small interfering RNA (siRNA), and antisense RNA (asRNA). Techniques that knock-out gene expression include clustered regularly interspaced short palindromic repeats (CRISPR), transcription activator-like effector nucleases (TALEN) and zinc-finger nucleases (ZFN). Cell lines thusly engineered to have reduced SHMT2 expression or an SHMT2 gene rendered inoperative in turn have reduced or eliminated SHMT2 enzymatic activity and by extension produce less formate. Engineered cells are assayed in fed-batch conditions for the growth, productivity and the level of formate accumulation in the extracellular milieu. Engineered cells grow to higher cell densities and produced higher levels of protein due to reduced production of formate.

### Example 6

Experiment to Determine the Gene Targets for Metabolically Engineering CHO Cells Towards Suppression of glycerol Production.

Goal

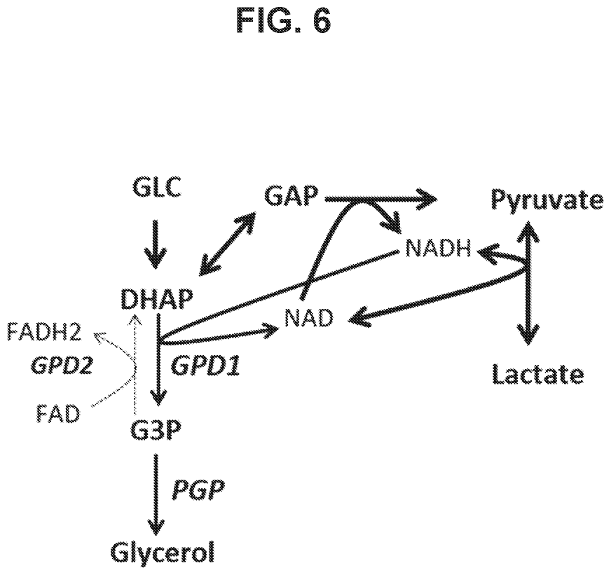

The cause for biosynthesis of the growth inhibitor glycerol in CHO cells was examined. Glycerol is known byproduct of glycerol-3-phosphate (FIG. 6). Recently, it has been reported that glycerol-3-phosphate is converted to glycerol by action of enzyme phosphoglycolate phosphatase (PGP) in pancreatic B cells and hepatocytes (Mugabo et. Al. 2016). Glycerol-3-phospate is product of enzymatic conversion of dihydroxyacetone phosphate (DHAP), a glycolytic intermediate, by cytosolic glycerol-3-phosphate dehydrogenase (GPD1) enzyme. Gene expression levels relevant enzymes in biosynthesis of glycerol were probed using RNAseq analysis. Based on the gene expression data, targets for metabolic engineering were identified in an effort to suppress the glycerol-3-phosphate flux towards production of glycerol.

Materials and Methods

A Pfizer proprietary RNAseq database was queried to provide a baseline expectation of gene expression levels, and by extension, the likely enzymatic activity of the key enzymes involved in the conversion of gycerol-3-phosphate to glycerol. The RNAseq data was derived from CHO cell line D which shares the same host as cell line C. Enzymes included in the study were GPD1, GPD2 and PGP. RNAseq data for B-actin was included for reference. Data was reported as reads per kilobase of transcript per million mapped reads (RPKM).

Results

The RNA seq data for key genes involved in glycerol-3-phosphate metabolism and hence glycerol accumulation are shown in Table 4. The genes PGP, GPD1, and GPD2 had a RPKM value between 5-21 suggesting expression of the genes (and potentially the corresponding protein) and the likelihood of enzymatic activity at this node in CHO cells. As the PGP gene is expressed, suppressing the activity of PGP enzyme genetically can potentially reduce glycerol production.

TABLE 4
RNAseq data for genes involved in glycerol biosynthesis. The
data are reported in reads per kilobase million (RPKM).

| Gene | RPKM |
|---|---|
| GPD1 | 13 |
| GPD2 | 21 |
| PGP | 5 |

### Example 7

Down-Regulation of PGP Gene Expression in CHO Cells to Suppress the Production of glycerol

The expression of PGP is targeted for down-regulation using molecular biology techniques that reduce gene expression (knock-down) or render the gene inoperative (knock-out). Techniques that knock-down gene expression, often referred to as RNA interference (RNAi), include micro RNA (miRNA), small interfering RNA (siRNA), and antisense RNA (asRNA). Techniques that knock-out gene expression include clustered regularly interspaced short palindromic repeats (CRISPR), transcription activator-like effector nucleases (TALEN) and zinc-finger nucleases (ZFN). Cell lines thusly engineered to have reduced PGP expression or a PGP gene rendered inoperative in turn have reduced or eliminated PGP enzymatic activity and by extension produce less glycerol. Engineered cells are assayed in fed-batch conditions for the growth, productivity and the level of glycerol accumulation in the extracellular milieu. Engineered cells grow to higher cell densities and produced higher levels of protein due to reduced production of glycerol.

### Example 8

Development of CHO Clones with Attenuated PGP Expression Levels

Background and Goal

Glycerol accumulates to high levels in cell culture media of fed-batch cultures, at times approaching approximately 19 mM by Day 14 in culture. Glycerol concentrations at and beyond 2 mM have been shown have growth inhibitory effects (FIG. 3A) and results in a wastage of nutrient carbon leading to an increase in osmolarity of the culture milieu. Suppression of glycerol production through the modulation of protein expression of key enzymes involved in glycerol producing pathways through knock down or knock out strategies was, therefore, attempted. Phosphoglycolate phosphatase (PGP) was hypothesized as an enzyme generating glycerol by hydrolysis of the phosphate group from glycerol-3-phosphate. The objective of this experiment was to develop stable CHO clonal cell lines with complete or partial knock-out (KO) of PGP gene using CRISPR CAS9 approach.

Materials and Methods

Guide RNAs (gRNAs) targeting exons of PGP were designed using a gRNA design algorithm. DNA constructs encoding the candidate gRNA sequences were co-transfected with Cas9 mRNA into a recombinant antibody producing CHO cell line (from here on referred to as wildtype (WT)) and gRNA efficacy evaluated using the GeneArt Genomic Cleavage Detection Assay. The two top performing gRNA were utilized to establish mutant clonal cell lines. Mutant clones were identified using Sanger sequencing to detect alterations in the PGP DNA sequence. Lysate was harvested from candidate mutant clones, from WT (untransfected) cell line, and from a control cell line that was transfected with gRNA/Cas9 but did not exhibit DNA alterations at the PGP locus. An SDS-PAGE gel was run using equal amounts of cell lysate and immunoblotting was performed to detect changes in PGP expression levels.

Results

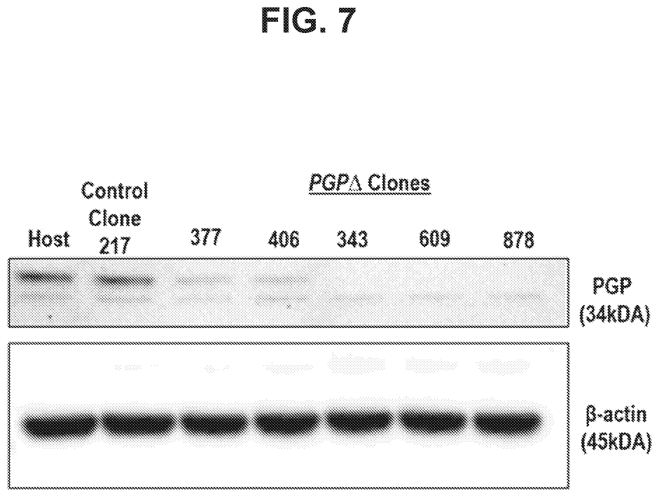

Stable knockout clones with reduced levels of PGP were generated through transfection of two independent gRNAs with Cas9 mRNAs (FIG. 7). Decreased PGP expression (FIG. 7, top band) was observed in two independent experiments in multiple mutant clonal cell lines. While residual PGP expression was observed in two of the generated clones (clones 377, 406) suggesting partial KO, PGP expression was undetectable in the lysate of other clones (clones 343, 609, 878) suggesting complete KO. The control clone 217 (which had been transfected with gRNA/Cas9 directed to PGP but did not exhibit DNA alterations at the PGP locus) and the wild-type host (untransfected) cell lines had greater PGP expression than any of the KO clones (FIG. 7)

### Example 9

Examination of Effect of Reduced PGP Protein Levels on glycerol Production in Fed-Batch Cultures

Background and Goal

The purpose of this experiment was to understand if reduction in PGP expression leads to a decrease in glycerol levels and glycerol production in fed batch cultures.

Materials and Methods

Wild-type host (WT), PGP knock-out (PGPΔ), and control clones as described in Example 8 were inoculated at densities of 1.0E6 cells/mL in ambr cell culture vessels. This system is intended to mimic bioreactor conditions on a smaller scale, where temperature, pH and nutrient supply can be well controlled. Cells were cultivated in this system for 12 days and were sampled on days 6, 8, and 11. Glycerol levels were measured using the BioHT Bioprocess Analyzer and specific glycerol production rates were calculated by monitoring changes in glycerol levels over time and normalizing to cell number.

Results

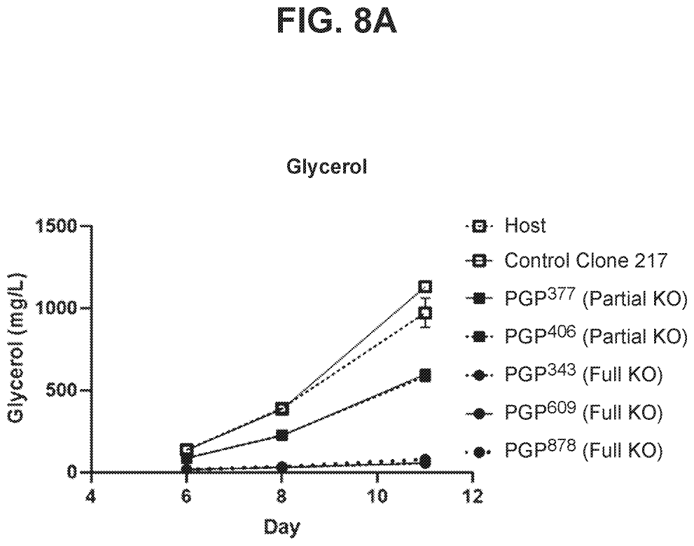

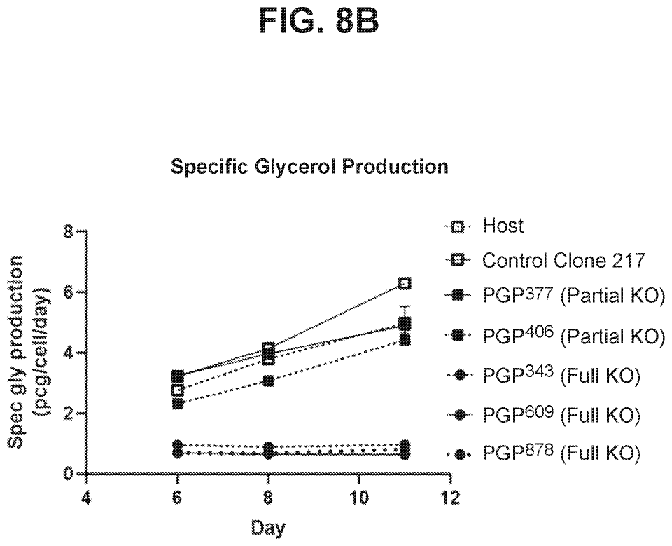

Spent media of PGPA cultures exhibited overall lower glycerol concentrations, as measured by both mg glycerol/liter (FIG. 8A) and specific glycerol production (picograms (pcg) per cell per day; FIG. 8B). Degree of glycerol reduction correlated with degree of PGP protein reduction as spent media from clones 343, 609, and 878 exhibited significantly lower levels of glycerol than that from clones 377, 406, control clone and WT (FIG. 8A). A decrease in specific glycerol production was also correlative with PGP expression levels in PGPΔ clones (FIG. 8B).

### Example 10

Examination of Effect of Reduced PGP Protein Levels on Accumulation of lactate in Fed-Batch Cultures.

Background and Goal

This experiment was performed to determine if reducing PGP protein expression levels would have an effect on lactate levels in cell culture media.

Materials and Methods

Wild-type host (WT), PGP knock-out (PGPA), and control clones as described in Examples 8 and 9 were inoculated at densities of 1.0E6 cells/mL in ambr vessels. This system is intended to mimic bioreactor conditions on a smaller scale, where temperature, pH and nutrient supply can be well controlled. Cells were monitored in this system for 12 days and sampled on days 1, 2, 4, 5, 6, 7, 8, 10, 11, and 12. Lactate levels were measured using BioProfile Nova Flex.

Results

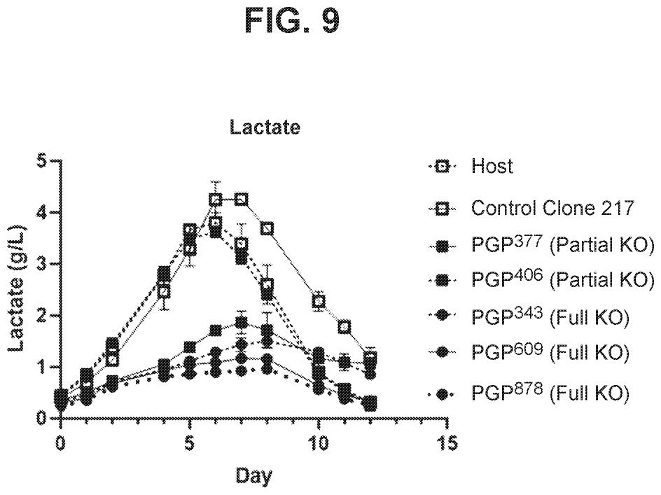

Reduced protein expression of PGP resulted in a decrease in lactate accumulation in cell culture milieu as seen in case of the complete KO PGPΔ clones (FIG. 9). The specific rate of lactate production was also low in these clones (data not shown).

### Example 11

Development of CHO Cell Lines with Attenuated SHMT2 Expression Levels

Background and Goal

Formate accumulates to high levels in cell culture media of fed batch cultures, approaching concentrations of approximately 6 mM by Day 8 in culture and at or beyond 10 mM by Day 14. Formate accumulation at and beyond 2 mM has been previously shown to have growth inhibitory effect (FIG. 3B). Inhibition of formate production through the modulation of protein expression of enzymes involved in carbon metabolism through knock down or knock out strategies was attempted in this experiment. The objective of this experiment was to develop stable CHO clone with reduced levels of SHMT2 protein expression. SHMT2 is a mitochondrial enzyme that catalyzes the cleavage of serine to glycine and methylene-tetrahydrofolate (THF). The enzymes MTHFD2/MTHFD2L convert methylene-THF to 10-formyl-THF which is then converted to formate by MTHFD1L.

Materials and Methods

Guide RNAs (gRNAs) targeting exons of SHMT2 were designed using a gRNA design algorithm. DNA constructs encoding the candidate gRNA sequences were co-transfected with Cas9 mRNA into a recombinant antibody producing CHO cell line (from here on referred to as wildtype (WT)) and gRNA efficacy evaluated using the GeneArt Genomic Cleavage Detection Assay. The two top performing gRNA were utilized to establish mutant clonal cell lines. Mutant clones were identified using Sanger sequencing to detect alterations in the SHMT2 DNA sequence. Lysate was harvested from candidate mutant clonal cell lines, from WT (untransfected) cell line, and from a control cell line that was transfected with gRNA/Cas9, but did not exhibit DNA alterations at the SHMT2 locus. An SDS-PAGE gel was run using equal amounts of cell lysate and immunoblotting was performed to detect changes in SHMT2 expression levels.

Results

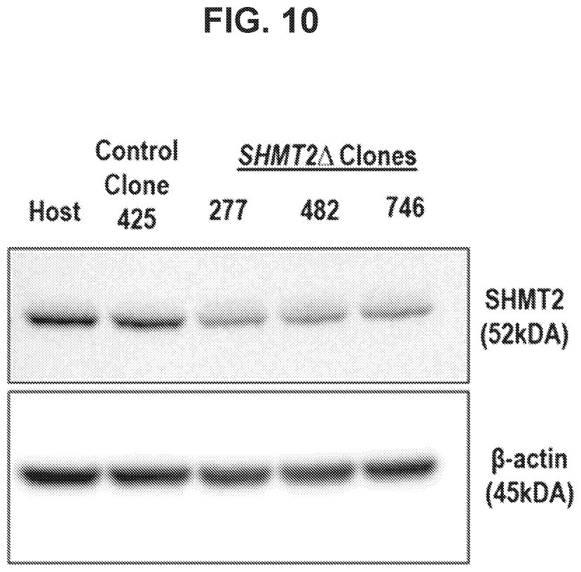

Clones with reduced protein levels of SHMT2 were generated through transfection of two independent gRNAs with Cas9 mRNAs (FIG. 10). Decreased SHMT2 protein expression was observed in two independent experiments in multiple mutant clonal cell lines suggesting partial KO in these clones (clones 482, 746 and 277). The control clone 425 (which had been transfected with gRNA/Cas9 directed to SHMT2 but did not exhibit DNA alterations at the SHMT2 locus) and the wild-type host (untransfected) cell lines had greater SHMT2 expression than any of the KO clones (FIG. 10)

### Example 12

Examination of Effect of Reduced SHMT2 Protein Levels on formate Production in Fed-Batch Cultures

Background and Goal

The purpose of this experiment was to understand if reduction in SHMT2 protein expression leads to a decrease in formate production and accumulation in fed-batch cultures.

Materials and Methods

Wild-type host (WT), SHMT2 knock-out (SHMT2Δ) and control CHO clones as described in Example 11 were inoculated at densities of 1.0E6 cells/mL in ambr vessels. This system is intended to mimic bioreactor conditions on a smaller scale, where temperature, pH and nutrient supply can be well controlled. Cells were monitored in this system for 12 days and sampled on days 6, 8 and 11. Formate levels were measured using the BioHT Bioprocess Analyzer and specific formate production rates were calculated by monitoring changes in formate levels over time and normalizing to cell number.

Results

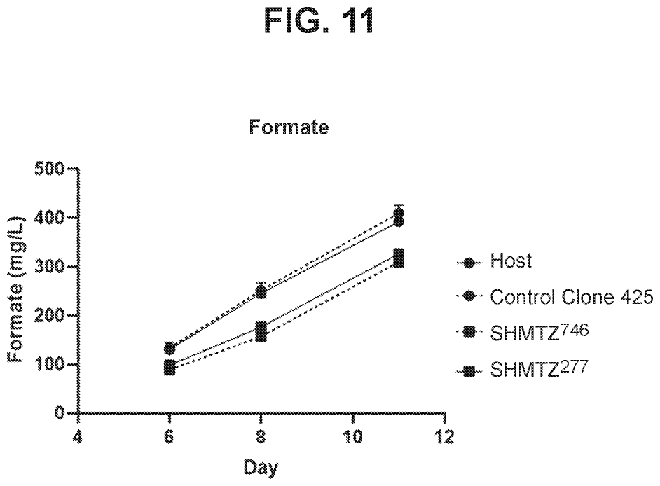

Spent media of SHMT2Δ cultures exhibited overall lower formate concentrations, as measured by mg formate/liter (FIG. 11).

### REFERENCES

Matthew Gagnon, Gregory Hiller, Yen-Tung Luan, Amy Kittredge, Jordy DeFelice, Denis Drapeau (2011) High-End pH-controlled delivery of glucose effectively suppresses lactate accumulation in CHO Fed-batch cultures. Biotechnology and Bioengineering 108(6):1328-1337

Yves Mugabo, Shangang Zhao, Annegrit Seifried, Sari Gezzar, Anfal Al-Mass, Dongwei Zhang, Julien Lamontagne, Camille Attane, Pegah Poursharifi, José Iglesias, Erik Joly, Marie-Line Peyot, Antje Gohla, S. R. Murthy Madiraju, and Marc Prentki (2016) Identification of a mammalian glycerol-3-phosphate phosphatase: Role in metabolism and signaling in pancreatic β-cells and hepatocytes. PNAS E430-439

1. A method of cell culture comprising (i) providing cells in a cell culture medium to start a cell culture process, wherein the cells are modified to reduce the level of synthesis of growth or productivity inhibitors by the cell, wherein the inhibitors are formate or glycerol.

2. The method of claim 1, wherein the inhibitor is formate, wherein the cells are modified to modify expression of one or more genes, and wherein the genes are selected from the group consisting of one or more of: serine hydroxymethyltransferase (cytosolic) (SHMT1), serine hydroxymethyltransferase (mitochondrial) (SHMT2), C-1-tetrahydrofolate synthase (cytoplasmic) (MTHFD1), monofunctional C-1-tetrahydrofolate synthase (mitochondrial) (MTHFD1L), bifunctional methylenetetrahydrofolate dehydrogenase/cyclohydrolase (mitochondrial) (MTHFD2), bifunctional methylenetetrahydrofolate dehydrogenase/cyclohydrolase 2-like (MTHFD2L), and mitochondrial folate transporter (SLC25A32).

3. The method of claim 1, wherein the inhibitor is glycerol, wherein the cells are modified to modify expression of one or more genes, and wherein the genes are selected from the group consisting of one or more of: glycerol-3-phosphate dehydrogenase (cytoplasmic) (GPD1), glycerol-3-phosphate dehydrogenase (mitochondrial) (GPD2), and phosphoglycolate phosphatase (PGP).

4. The method of claim 2, wherein the one or more genes are modified to decrease gene expression.

5. The method of claim 1, wherein the method further comprises (ii) maintaining the inhibitor formate or glycerol in the cell culture medium (a) at a concentration of no more than C2 or (b) at a concentration of at least C1 and no more than C2.

6. The method of claim 5, wherein the inhibitor is formate, wherein C1 is 0.001 mM, and wherein C2 is 20 mM, 15, mM, 10 mM, 8 mM 6 mM, 4 mM, or 2 mM.

7. The method of claim 5, wherein the inhibitor is glycerol, wherein C1 is 0.001 mM, and wherein C2 is 20 mM, 15, mM, 10 mM, 8 mM 6 mM, 4 mM, or 2 mM.

8. The method of claim 5, wherein step (ii) comprises the step of measuring the concentration of formate or glycerol, and,
a) when the measured concentration is above C2, the concentration of a precursor of formate or glycerol in the cell culture medium is decreased by reducing the amount of precursor of formate or glycerol, respectively, provided to the cell culture medium, or
b) when the measured concentration is below C1, the concentration of formate or glycerol in the cell culture medium is increased by adding formate or glycerol, respectively to the cell culture medium.

9. The method of claim 8, wherein the concentration of formate or glycerol is measured using NMR, HPLC or UPLC, optionally online.

10. The method of claim 1, wherein a pH sensor is used to monitor pH of the cell culture, and, in response to a rise above a predetermined pH value, glucose is fed to the cell culture.

11. The method of claim 1, wherein the cell culture is a fed batch culture.

12. The method of claim 5, wherein the cell culture method comprises a growth phase and a production phase and step (ii) is applied during the growth phase.

13. The method of claim 1, wherein modifying the expression of the one or more genes comprises:
(a) gene deletion, disruption, substitution, point mutation, multiple point mutation, insertion mutation or frameshift mutation of the gene, or
(b) introduction of one or more nucleic acids comprising the one or more genes into the cell, optionally as an expressible construct or expressible vector construct.

14. A cell comprising one or more modified genes which reduces the level of synthesis of growth or productivity inhibitors by the cell, wherein the inhibitors are formate or glycerol.

15. The cell of claim 14, wherein the inhibitor is formate, wherein the cell is modified to modify expression of one or more genes, and wherein the genes are selected from the group consisting of one or more of: serine hydroxymethyltransferase (cytosolic) (SHMT1), serine hydroxymethyltransferase (mitochondrial) (SHMT2), C-1-tetrahydrofolate synthase (cytoplasmic) (MTHFD1), monofunctional C-1-tetrahydrofolate synthase (mitochondrial) (MTHFD1L), bifunctional methylenetetrahydrofolate dehydrogenase/cyclohydrolase (mitochondrial) (MTHFD2), bifunctional methylenetetrahydrofolate dehydrogenase/cyclohydrolase 2-like (MTHFD2L), and mitochondrial folate transporter (SLC25A32).

16. The cell of claim 14, wherein the inhibitor is glycerol, wherein the cell is modified to modify expression of one or more genes, and wherein the genes are selected from the group consisting of one or more of: glycerol-3-phosphate dehydrogenase (cytoplasmic) (GPD1), glycerol-3-phosphate dehydrogenase (mitochondrial) (GPD2), and phosphoglycolate phosphatase (PGP).

17. The cell of claim 14, wherein the one or more genes are modified to decrease gene expression, and wherein gene deletion, disruption, substitution, point mutation, multiple point mutation, insertion mutation or frameshift mutation is applied to the modified gene.

18. The cell according to claim 17 wherein the decreased gene expression results in less than or equal to any of, 90, 80, 70, 60, 50, 45, 40, 35, 30, 25, 20, 15, 10, 5, 2.5 percent level of the expression or activity of the respective modified gene as compared to the activity of the gene in a corresponding unmodified cell.

19. The cell of claim 14, wherein the cells are CHO cells.

20. (canceled)

21. (canceled)

22. The method of claim 1, wherein cell growth or productivity are increased as compared to a control culture, said control culture being identical except comprising unmodified cells.

23-25. (canceled)
